(19) United States
(12) Patent Application Publication
(10) Pub. No.: US 2017/0322204 A1
(43) Pub. Date: Nov. 9, 2017

(54) SIMULTANEOUS DETECTION OF BIOMOLECULES IN BIOLOGICAL ENTITIES

(71) Applicants: MORPHOSYS AG, Planegg (DE); ALBERT-LUDWIGS-UNIVERSITAET FREIBURG, Villingen-Schwenningen (DE)

(72) Inventors: Markus Enzelberger, Planegg-Martinsried (DE); Andreas Boll, Munich (DE); Beate Diefenbach-Streiber, Windbach (DE); Guenter Roth, 15660396 (DE); Felix von Stetten, Freiburg-Tiengen (DE); Fabian Stumpf, Freiburg (DE)

(21) Appl. No.: 15/660,396

(22) Filed: Jul. 26, 2017

Related U.S. Application Data
(63) Continuation of application No. 13/989,814, filed on Aug. 29, 2013, now abandoned.
(60) Provisional application No. 61/418,423, filed on Dec. 1, 2010.

(30) Foreign Application Priority Data
Dec. 1, 2010 (EP) 10193291.1

(51) Int. Cl. G01N 33/53; G01N 33/68; G01N 33/543; C12Q 1/68

(52) U.S. Cl. CPC G01N 33/5308; C12Q 1/6869; C12Q 1/6834; G01N 33/543; G01N 33/6842; C12Q 2523/303; C12Q 2527/109; C12Q 2527/119; C12Q 2563/149; C12Q 2563/159; C12Q 2531/113

(57) ABSTRACT

The present invention provides methods, immunoassays, kits and devices pertaining to the detection of multiple biomolecules from single cells or other biological entities. It also enables the highly parallel detection of interacting biomolecules from such entities.

### INTRODUCTION

This application is a continuation of U.S. Ser. No. 13/989,814 filed Aug. 29, 2013, which is the U.S. National Stage of PCT/EP2011/071433 filed Nov. 30, 2011, which claims the benefit of priority from EP 10193291.1 filed Dec. 1, 2010 and U.S. Provisional Ser. No. 61/418,423 filed Dec. 1, 2010, each of which are incorporated by reference in their entireties.

### FIELD OF THE INVENTION

The present invention provides methods, immunoassays, kits and devices pertaining to the detection of multiple biomolecules from single cells or other biological entities. It also enables the highly parallel detection of biomolecules from such entities.

### BACKGROUND OF THE INVENTION

The living world is composed of various types of organic and inorganic matter, including nucleic acids, polypeptides, carbohydrates, fatty acids and many more. They form cells, tissue, organs and organisms which, in turn, react to and interact with substances and compounds present in other compartments or the surrounding tissue or liquids. For all these substances (referred to as “biounits” or “biomolecules” in the present invention) detection methods do exist. The best suited detection method for a given problem depends on many factors, such as the nature of the biounit itself and the origin of the sample to be tested. Overall, in the last decade and years the sensitivity of most detection methods has greatly improved. For certain biounits detection methods exist which even enable the detection of single molecules. Such sensitive methods often require complex processing of the samples, in order to eliminate factors that might interfere with the respective detection method. The present invention discloses novel and superior methods for the detection of biounits. Such biounits may, for example, be biomolecules.

The biounits or biomolecules that are detected or identified with the methods of the present invention are comprised in a sample and the present invention achieves the parallel detection of at least two of said biounits or biomolecules. The sample itself may comprise one or more (typically more) structural or biological subunits (“biological entities” in the terminology of the present invention) containing the biounits or biomolecules. As an example: the sample may be a drop of blood, the biological entity one single B cell comprised in said drop of blood, and the biounits which are detected in parallel are the nucleic acids encoding the VH and the VL chain of the antibody produced by said B cell.

Theoretically the presence of several different single (or a very few) biounits or biomolecules in a biological entity, such as a cell, can be analyzed (e.g., the presence or absence of a given gene or polypeptide in a cell) by separating the biological entity or cell into different batches of biounits/biomolecules and identifying in each batch the desired biounit/biomolecule of interest. Such separation and detection methods are however very cumbersome, in particular if more than one, potentially even hundreds or thousands, of biological entities are to be analyzed in a sample. Therefore the process has to be performed in parallel instead of sequentially. Many existing detection systems fail in a parallel approach. Also, parallel approaches focus on the parallel detection of one biounit or biomolecule per biological entity or cell.

For a highly parallel processing and parallel detection of at least two biounits or biomolecules per biological entity or cell, the sample, prior to detection, has to be divided by transferring each biological entity or cell, into an individual compartment or cavity (referred to as “effective range” or “compartment” in the present invention). In particular, if several biounits or biomolecules have to be detected in a single sample then the location information needs to be preserved, i.e. the information which biounit/biomolecule is present in which biological entity. This can, for example, be achieved by physically splitting up or dividing the sample into different containers or cavities, such as Eppendorf tubes or the wells of a 96-well or a 384-well microtiter plate or the cavities of a picotiterplate, and depends on the nature of the sample, the biological entity or cell, and the biounit/biomolecule to be detected. But such system lack the capability of highly parallel processing. A higher parallelization can be achieved in so called picotiterplates containing ˜10⁶ or even more cavities. But here the distribution of the sample is in particular troublesome if the sample has only a small volume, e.g., only a few microlitres or even picolitres, and the volume has to be further decreased via the splitting process. Obviously this leads to problems, in particular if the sample itself only comprises very few of the biounits/biomolecules to be tested. Due to the statistical distribution of the biounits/biomolecules in the splitting process, the individual cavities might not contain any of the biounits/biomolecules, or only at a concentration below the detection threshold. This leads to obvious problems if single biounits/biomolecules are to be detected. Another problem is the practical difficulty to handle small volumes, which tend to dry out and also show a tendency to stick to the surface of the cavity and are not affected by gravity. On the other hand, if the sample contains many biounits/biomolecules, the splitting process, if done statistically, will destroy the location information between biounits/biomolecules. This is for example the case in the second generation sequencing technologies, where DNA is taken from several cells and mixed before amplification. In other aspects of the invention the sample may contain biological entities which are larger than the compartment (e.g., if the compartment is formed by the cavities of a picotiterplate). For example, the sample may be a kidney, or a part thereof, and the biological entities may be nephrons. In such cases, the present invention provides a highly parallel way has to isolate the biounits/biomolecules and detect them in parallel.

One problem solved by the present invention is the highly parallel processing of the biological entities or cells and the simultaneous analysis of two or more biounits or biomolecules that are present in or originate from said biological entity or cell. The two or more biounits or biomolecules that are analyzed and detected in the methods of the present invention interact with each other in any way or form. This could for example be by directly binding to each other. Alternatively they can bind to a third protein or other entity or moiety which “connects” the two biounits or biomolecules. Yet alternatively, the two or more biounits or biomolecules could also simply be present in the same biological entity or cell with any direct or indirect interaction. In such cases the present invention can be applied to detect such two or more biounits or biomolecules present in a given biological entity or cell. In certain embodiments the two or more biounits or biomolecules are not analyzed immediately, but are stored for later analysis. This is achieved by providing a moiety which is able to bind to the biounits or biomolecules, or to derivatives generated from said biounits or biomolecules.

One step towards the solution to said problems is the use of emulsions, e.g., water in oil emulsions. Here, typically small aqueous droplets are surrounded by oil, thereby generating an effective range capable of entrapping single biological entities, such as single cells. The size of the droplets can be so large to even include complete tissues or functional units like for example nephrons from the kidney. Biounits or biomolecules that are in relation or proximity to each other (e.g., present in the same cell) or that interact with each other in any way or form will be captured and become entrapped by one single droplet of the emulsion. In contrast, biounits/biomolecules which are comprised in separate biological entities or cells will be separated. Practically it is very difficult to generate such emulsions, in particular, emulsions which are amenable for further processing steps and which only contain a single biological entity. One drawback with aqueous droplets is that they only allow for certain types of processing. In cases where the biounits, biomolecules or biological entities are nucleic acids they can be amplified via PCR or RT-PCR. Cells can be multiplied by regular growth of the cells. However such technologies, (e.g., the RainDance technology, see PNAS (2009) 106, 14195-200), do not provide or disclose any moieties which are able to bind biounits, biomolecules or derivatives thereof. This is a prerequisite for subsequent high parallel analysis, e.g., sequencing.

Rettig & Folch disclose a method which enables the entrapment of single cells in microwell arrays (Anal Chem (2005) 77, 5628-34). Related to this, BioTrove, Inc. (now part of Life Technologies Corporation) disclosed a method to entrap single cells into as much as 20,000 wells or more of an appropriately designed chip (the Living Chip™). These methods however only pertain to the problem of the physical distribution of biounits/biomolecules into different cavities. It is completely silent about any potential uses with respect to the present invention, e.g., the entrapment of related or interacting biounits in a storage, or the identification and/or detection of the interacting partners.

Grosvenor et al. (Anal Chem (2000) 72, 2590-4) report on the development of certain assays in picoliter format. This publication however relates only to assays in small scale per se. The assays performed do not utilize whole cells and they are simple in the sense that they do not allow for any major manipulation steps, such as e.g., sequencing. Curnow et al. (Invest Opthalmol Visual Sci (2005) 46, 4251-9) describe a multiplex assay on beads utilizing cells. They did not however analyze individual cells or biological entities. A similar method is disclosed in Vignali (J Immunol Meth (2000) 243, 243-55). Taniguchi et al (Nature Methods (2009) 6, 503-6) describe a PCR approach in which the expression of several genes of a single cell can be analyzed. This approach is however very cumbersome and furthermore limited to the analysis of single cells, rather than an entire population of cells or the detection and/or identification of connected or interacting biounits/biomolecules within said larger units.

Other reports focus on sequencing technologies and related application, such as for example the detection of polymorphisms (see e.g., WO/2005/082098, U.S. Pat. No. 6,013,445, US 2009/0269749, US 2006/0292611, US 2006/0228721 or U.S. Pat. No. 7,323,305), but none of these reports aims for or accomplishes the simultaneous detection or identification of biounits or biomolecules, such as nucleic acids from biological entities or cells, as contemplated in the present invention.

Zeng et al. (Anal Chem (2010) 82, 3183-90) disclose microfluidic arrays which are suitable for single cell genetic analysis. They describe a multiplex single cell PCR method which was developed to detect and quantify wild type cells and/or mutant/pathogenic cells. Zeng et al. utilize microbeads which are functionalized with multiple forward primers, i.e. primers which are specific for the respective wild type gene or the mutant gene. In contrast to Zeng et al., the present invention does not only detect one biounit or biomolecule per cell, i.e. the wild type or the mutant version of a gene, but is able to detect two or more biomolecules per biological entity or cell. Furthermore, the method describe in Zeng et al. is only technically possible with long genomic DNA molecules, but not for any other biounits or biomolecules.

US 2006/0263836 describes a system for multiplexed microparticle-based. The assay system however fundamentally differs from the methods of the present invention in that only one biounuit/biomolecule is detected in the biological entity or sample of US 2006/0263836 (namely an antibody). The second biounit/biomolecule which is utilized in the assay is an antigen which immobilizes on the microparticles, i.e. said second biomolecule is not a biomolecule which is derived or contained in the biological entity or sample, but is artificially added during the steps of the assay disclosed in US 2006/0263836.

WO 2007/081387 describes methods and assays for the identification of interactions between certain biounits and biomolecules. Likewise, one of the biounits/biomolecules used in the methods of WO 2007/081387 is not a biomolecule which is derived or contained in the biological entity or sample, but is artificially added during the steps of the assay. In other words, one of the biounits/biomolecules in WO 2007/081387 is used to make a logical connection to the second biounit/biomolecule, knowledge about the interaction to be detected is already required.

US 2005/0227264 describes a nucleic acid amplification method in a water-in-oil emulsion in a continuous flow system. Apart from the fact that the utility of this method is an entirely different one compared to the present invention, US 2005/0227264 merely aims to encode and decode individual PCR products by way of the disclosed PCT technique.

U.S. Pat. No. 7,244,567 discloses a method of sequencing the sense and the antisense strand of a nucleic acid molecule at the same time by using a technology to temporarily block one of the sequencing primers. U.S. Pat. No. 7,244,567 does however not disclose the concept of spatially separating the biological entity or cell prior to further processing of the sample. Furthermore, U.S. Pat. No. 7,244,567 sequences one single nucleic acid molecules from both ends and hence does not detect or identify two or more biounits or biomolecules.

In summary, all methods recited above suffer from one or more disadvantages or restrictions. The physical splitting of samples of small volumes is an inherent problem of single cell analysis. Methods utilizing small aqueous solutions, such as emulsions, suffer from a lack of a component which enables the storage and subsequent analysis of the related, interacting or connected biounits. Available methods for the simultaneous analysis, e.g., sequencing, of biounits suffer from technological pitfalls, such as inefficiency (e.g., linkage PCR, a method which cross-links two different nucleic acid molecules via a PCR-based amplification reaction, has never been efficiently demonstrated in emulsions) or lack of robustness, e.g., a low signal-to-noise ratio, or even results which are qualitative rather than quantitative. For example, the linkage PCR performed in WO 2005/042774 (Symphogen) requires two PCR reactions, and the PCR products are identified by classical DNA sequencing. The present invention achieves the same result in a single PCR step, wherein this PCR reaction already enables direct sequencing. This difference leads to a completely different throughput, enabling the large scale processing of samples and subsequent identification of the respective PCR products.

The detection and/or identification of the at least two biounits or biomolecules of the present invention is achieved by the entrapment of the biological entity or cell comprising said at least two biounits or biomolecules in an effective range or compartment. It might be advantageous if the compartment comprises a moiety which is able to bind the biounits or the biomolecules, or derivatives of said biounits or biomolecules. The entrapment of the biological entity or the cell in the compartment ensures that the information is preserved that the biounits or the biomolecules have a common origin or interact with each other in the sample, e.g., via a temporal interaction. This information, also referred to as the location information, can then be transferred or copied onto a moiety which is able to bind the biounits or the biomolecules, or the derivatives of said biounits or biomolecules. The preservation of the location information also ensures that no biounits or biomolecules are present in a compartment which is not part of or comprised in said biological entity or cell, i.e. no false-positive biounits are detected or identified. This is not possible with technologies known in the art.

With current technologies it is therefore not possible to reliably and highly parallel detect, identify, register, and/or quantify the relation, interaction or common origin of two or more related or connected biounits or biomolecules, e.g., two or more genes within each cell over a large population of cells. Currently available single cell technologies only allow the analysis and detection of one or several single biounits or biomolecules. The present invention overcomes this limitation and simplifies the handling of biounit or biomolecules populations in any size. One of its uses is the registration or detection of two mRNAs originating from a single cell, and their identification by massively parallel sequencing in the dimension of entire cell populations.

In order to achieve this effect the present invention makes use of a storage. The storage is a moiety which binds to the biounits or the biomolecules which are detected in the present invention. It stores the biounits or biomolecules which are entrapped and spatially separated in a certain location, compartment or effective range, thereby causing a physical connection of the biounits or biomolecules to be detected. The togetherness of the detected biomolecules or biounits on the moiety which is able to bind said biomolecules, biounits or derivatives thereof, is equal to the togetherness of the biomolecules or biounits in the biological entity or cell in the beginning. Only biounits or biomolecules so entrapped or fixed in a respective compartment are amenable for subsequent highly parallel processing of the respective molecules, and ultimately their detection and/or identification.

### Definitions

The term “biounit” refers to any molecule, an assembly or complex of molecules or a cell (or a subunit thereof).. The term biounit also includes tissues, organs, cell organelles, entire organisms or any other entity which is part of or which is comprised in a biological system.

The term biounit includes biomolecules. The term “biomolecule” is art recognized and includes any molecule which is or can be generated by a biological system. Biomolecules include molecules which are composed of amino acids (such as polypeptides, proteins or peptides), nucleotides (such as DNA, RNA or modifications thereof), carbohydrates (or any other form of sugars), or fatty acids, lipids, or naturally occurring small organic molecules, metabolites, or any derivatives, parts or combinations of any of the foregoing.

Antibodies and antibody fragments are exemplary and preferred polypeptides of the present invention. Exemplary nucleic acids include the genes encoding antibodies and antibody fragments. Biounits may exist in nature or may be derived from molecules that exist in nature. The term biounit also includes molecules that are exogenous to a given biological system, but which were added to the same in order to, for example, achieve or study a certain effect in said biological system. A pharmaceutically active compound which is administered or brought into contact with a certain cell, tissue or patient is therefore a biounit according to the present invention. Biounits and biomolecules may also be metabolites, e.g., anabolic or catabolic metabolic molecules. Two (or more) genes or gene products which are encoded on a (single) genome are considered as two (or more) biounits or biomolecules. Biounits and biomolecules may be comprised in, on or in association with a biological entity. Under certain circumstances they may be a biological entity on their own. A biounit or biomolecules may be a binder, a binder target, a modifieror a modifiertarget or a direct or indirect derivative thereof.

The present invention provides methods for the highly parallel detection of at least two biounits or biomolecules, such as nucleic acid or polypeptides from a sample. In particular, and preferably, said biounits or biomolecules interact with each other by any shape, form or means. Such interaction, relationship or connection is characterized by the term “interact” or “interaction”. An interaction may be a physical interaction, which may be covalent or non-covalent in nature, but an interaction may also be another non-physical logical connection between two biomolecules or biounits. Examples include:
a current or past interaction between at least two biounits, such as
   the interaction between an antigen or an antibody,
   a hybridization between two DNA molecules
   a linkage between two DNA molecules or genes
   the interaction between a virus and a cell
   the interaction between two cells
   the interaction or binding of two antibodies with the same antigen
a common origin of the at least two biounits, such as
   two molecules which are present in the same cell
   two messenger RNAs which are derived or originating from the same genome
   two DNA sequences, RNAs, peptides or proteins derived or originating from the same genome or cell
   two daughter cells
   cells originating from the same ancestor.

In certain aspects the present invention provides a method for the detection of two or more biomolecules or biounits in a biological entity or a cell. In alternative aspects the present invention provides a method for the detection at least two biomolecules or biounits in a biological entity or a cell. In certain aspects more than two, for example three, four, five, ten, twenty or even more biomolecules or biounits are detected with the present invention.

The term “binder” refers to a biounit or biomolecule which is capable of binding to another biounit or biomolecule (referred to as the “binder target”). Binders and binder targets of the present invention may be any biounit or biomolecule as defined herein above. Typical binders of the present invention include antibodies, as well as derivatives thereof. Typical binder targets of the present invention include antigens. Most commonly antigens are of proteinaceous structure, but antigens may also be of different nature like for example carbohydrates, fatty acids or lipids. The storage and the binder can also be the same entity, i.e. in certain embodiments the same molecule may serve as a binder and a storage in accordance with the present invention.

The term “modifier” refers to a biounit or biomolecule which is able to modify another biounit or biomolecule (referred to as the “modifier target”). Modifiers includes enzymes (for example phosphatases) which add certain moieties (for example a phosphate group or a sugar moiety) to a substrate, e.g., a binder, thereby increasing or decreasing the binding activity of the binder or change the targeting, the physical, physiological or chemical properties of a binder (i.e. a modifier target in the terminology of the present invention).

The term “replicate” refers to a molecule which is derived from or which is generated from a source molecule. The replicate can be an exact copy of the source molecule, such as for example a double-stranded deoxyribonucleic acid molecule which has been generated by replication of a source deoxyribonucleic acid molecule. The replicate can also be a derivative of the source molecule, such as for example a messenger RNA, which was generated by transcription of a source deoxyribonucleic acid molecule. In the latter case the replicate still carries the information from which source molecule it is derived, i.e. it is still possible to unambiguous back track or identify the source molecule.

The term “derivative” refers to a molecule which is a derivate, a copy or an image of another (source) molecule in the sense that it is a direct and unambiguous product of the source molecule, i.e. the exact identity and nature of the source molecule is known or can be deduced if the derivative is known. PCR products are typical derivatives in accordance with the present invention. Identity, nature (and sequence) of the source molecule can immediately be deduced from a given PCR product. Likewise, RT-PCR products are derivatives in accordance with the present invention, i.e. cDNA re-scripts of mRNA strands synthesized via reverse transcription are derivatives.

The term “biological entity” refers to a functional biological unit which comprises at least two biounits or biomolecules which are both present inside, on or near to said biological unit, which are attached to it, which interact with or bind to each other, which interact with or bind to another third molecule or which together cause a certain downstream event or have some other causal relationship. In its easiest form such biological entity encompasses a molecule which interacts or binds to another molecule, such as a binder and the corresponding binder target. Examples of biological entities are an antibody and its corresponding antigen, a ligand and a receptor, an enzyme and its substrate, or a drug and its drug target. In another form the biological entity encompasses a molecule which modifies another molecule, such as a modifier and the corresponding modifier target. Examples are an enzyme, such as a phosphatase, which modifies (in this case phosphorylates) a target molecule. Other biological entities are molecules complexes which consist of or comprise two or more biounits or biomolecules. Such biological entities may be protein/polypeptide, RNA and/or DNA complexes, homo- or heteromeric protein or enzyme complexes such as antibodies or ribosomes. Other biological entities are single cells, viruses, bacteria, cell compartments, cell clusters, tissue, organs or multicellular organisms. The at least biounits in a biological entity interact within the biological entity in any shape or form.

The term “storage” refers to a moiety which comprises a binder for the at least two biounits or biomolecules, or derivatives or replicates thereof. In certain embodiments of the present invention the storage itself is able or has the capability to bind the biounits or biomolecules of the present invention, or any derivatives or replicates thereof. In certain preferred embodiments, the storage comprises moieties which are able to bind the derivatives of said biounits or biomolecules. The role of the storage or the moiety is to absorb and trap the biounits or biomolecules in the effective range (qualitatively, more preferably quantitatively), thereby causing a physical connection, enabling further processing of the stored biounits or biomolecules (e.g., replication and/or modification), and ultimately their detection. Examples include a bead, a glass slide, a microtiterplate, a picotiterplate, or a lid of any of the foregoing. Detection and identification of the stored biounits or biomolecules may occur in different ways and depends on the nature of the biounits or biomolecules. Examples include sequencing of DNA, RT-PCR of RNA, measuring the biological activity of enzymes, determining the binding characteristics of antibodies or measuring the infectivity of phages or bacteria. Sequencing can be performed directly on the stored biounits or biomolecules, or on derivatives of the biounits or biomolecules. Appropriate primers may be added for the sequencing step. The sequencing primers and the sequencing reaction for the detection and the identification of the biounits or biomolecules may be performed simultaneously or subsequently. Under certain circumstances it may be advantageous to perform the sequencing reaction subsequently, i.e. to first add the first sequencing primer and perform the first sequencing reaction and then, subsequently, add the second sequencing primer and perform the second sequencing reaction.

Under certain circumstances, the effective range, the compartment or the biological entity itself can also act as storage. For example, the biological entity may act as the storage, if the biounits can be directly linked to the biological entity. This can for example be achieved via cross-linking molecules, e.g., polymerization or polycondensation, such as via formaldehyde or drying. The effective range or compartment may also act as the storage. For example, if the effective range or compartment is a liposome or a well, then the wall of the liposome or the well could act as storage. The storage and the binder can also be the same entity, i.e. in certain embodiments of the present invention the same molecule may serve as a binder and a storage in accordance with the present invention.

In certain embodiments of the present invention the biounits, biomolecules or derivatives thereof are nucleic acids that bind by hybridization to a moiety which is able to bind said nucleic acid, wherein said moiety is a solid-phase particle. In certain embodiments said solid-phase particle is used for sequencing in step (e).

In certain embodiments of the present invention, said biounits, biomolecules or derivatives thereof are polypeptides or proteins that bind directly to the surface of the moiety which is able to bind said polypeptides or proteins, wherein said moiety is a solid-phase particle, and wherein said biounits, biomolecules or derivatives on said solid-phase particle are detected via an immunoassay.

The term “effective range” refers to both, a location or spatial range in which a biochemical or a chemical reaction occurs, or a location or spatial range which comprises at least two biounits or biomolecules. The biounits or biomolecules are trapped in the effective range and can for example be modified or bound in said effective range (they can e.g., act as binder targets or as modifier targets). The size of the effective range can change over time and furthermore can increased, decreased or stabilized through internal or external parameters. For certain biochemical reactions it is preferable to utilize very small effective ranges. The effective range can be formed by any means appropriate to keep the biounits or biomolecules trapped within it. Examples are the walls of the wells of a microtiterplate or any other physical means, a current or an electric charge. An effective range which is generated by a physical means is referred to as “compartment”. An effective range or compartment may or may not comprise a storage. In certain embodiments of the present invention, a storage may not be required since the biounits or biomolecules (or their derivatives) which are entrapped in the effective range or compartment can be detected or identified directly, i.e. there is no need to further process the biounits or biomolecules. In alternative embodiments, a storage may be required since the subsequent processing of the biounits or biomolecules (or their derivatives) requires that the biounits or bimolecules are kept together and remain entrapped within the effective range or the compartment. The process of bringing or transferring the biounits or biomolecules of the present invention into an effective range or a compartment is referred to as “entrapping” or “spatially separating” the respective biounits or biomolecules.

In certain embodiments of the present invention, the biological entity comprising two interaction biounits or biomolecules, is spatially separated from the other biological entities comprised in the same sample. In certain embodiments said biological entity is a cell, preferably a single cell, such as a B cell. In certain embodiments of the present invention, a cell comprising two interaction biounits or biomolecules, is spatially separated from the other cells comprised in the same sample. In certain embodiments said cell is a single cell, such as a B cell.

The term “sample” refers to any material that comprises at least one biological entity or cell. Very often a sample comprises more than one, sometimes even thousand, millions or even more biological entities or cells. For example a sample of 1 ml of blood contains more than 4 billion of cells, i.e. more than 4 billion biological entities, and each biological entity comprises thousands of different biounits or biomolecules.

The term “location information” is the information which tells if certain biounits or biomolecules are contained in, are derived from or are connected from or to the same biological entity or cell within a sample.

The term “tag” as used herein refers to any peptide sequence suitable for purification or identification of molecules. A tag specifically binds to another moiety with affinity for the tag. Such moieties which specifically bind to a tag are usually attached to a matrix or a resin, such as agarose beads. Moieties which specifically bind to tags include antibodies, nickel or cobalt ions or resins, biotin, amylose, maltose, and cyclodextrin. Exemplary purification tags include histidine tags (such as a hexahistidine peptide), which will bind to metal ions such as nickel or cobalt ions. The term “tag” also includes “epitope tags”, i.e. peptide sequences which are specifically recognized by antibodies. Exemplary epitope tags include the FLAG tag, which is specifically recognized by a monoclonal anti-FLAG antibody. The peptide sequence recognized by the anti-FLAG antibody consists of the sequence DYKDDDDK or a substantially identical variant thereof. Therefore, in certain embodiments the purification tag comprises or consists of a peptide sequence which is specifically recognized by an antibody.

The term “simultaneous” or “simultaneously” in accordance with the present invention refers to detection of the at least two biounits or biomolecules from a single sample. Said at least biounits are biosamples are entrapped in an effective range or compartment to preserve the location information. This makes possible the highly parallel detection of the at least two biounits or biomolecules in a single sample or in a single biological entity or compartment.

The term “detect” or “detection” is art recognized and refers to the identification of known biounits or biomolecules in a given sample, biological entity or compartment.

The term “identify” or “identification” is also art recognized and refers to the identification of biounits or biomolecules in a given sample, biological entity or compartment, wherein the presence of said biounits or biomolecules was not known or merely suspected.

### BRIEF DESCRIPTION OF DRAWINGS

FIG. 1 illustrates the method steps of one of the embodiments of the present invention. The meaning of the symbols and structures is shown on the top of the figure. The “biounit” in the figure may also be a “biomolecule”, the “biological entity” may also be a “cell”, and the “effective range” may also be a “compartment”, all terms as defined herein above. FIGS. 1—A.1 refers to a scenario wherein two biounits or biomolecules interact with each other, thereby forming a biological entity. FIGS. 1—A.2 refers to a scenario wherein the two biounits or biomolecules are from the same origin or biological entity, e.g., the same cell, without a direct interaction. In step B the biounits or biomolecules are entrapped in an effective range or compartment. In FIGS. 1—B.1 the binders for the biounits or biomolecules are located on the biological entity or the cell. In. FIGS. 1—B.2 the binders for the biounits or biomolecules are located on the effective range or compartment. In FIGS. 1—B.3 the binders for the biounits or biomolecules additionally comprise a storage or a moiety which is able to bind the biounits, the biomolecules or derivatives thereof. In certain embodiments, the binder and the storage may also be the same entity or molecule. In step C the biounits or biomolecules are released from the biological entity or cell, but are still in spatial proximity to the binder since they are entrapped in an effective range or compartment. Scenario B.1 leads to the situation depicted in FIGS. 1—C.1, scenario B.2 to the situation depicted in FIGS. 1—C.2 and scenario B.3 to the situation depicted in FIGS. 1—C.3. In step D the biounits or biomolecules bind to the respective binders under appropriate conditions. Scenario C.1 leads to the situation depicted in FIGS. 1—D.1, scenario C.2 to the situation depicted in FIGS. 1—D.2 and scenario C.3 to the situation depicted in FIGS. 1—D.3. In step E the biounits or biomolecules are detected or identified by appropriate means. In FIGS. 1—E.1 the biounits or biomolecules are detected or identified within the effective range or compartment. In FIGS. 1—E.2 the biounits or biomolecules are detected or identified within the effective range or compartment while bound on the storage or on a moiety which is able to bind the biounits, biomolecules or derivatives thereof. In FIGS. 1—E.3 the biounits or biomolecules are detected or identified outside or without the effective range or compartment. The effective range or compartment is no longer needed since the biounits or biomolecules are bound on the storage or on a moiety which is able to bind the biounits, biomolecules or derivatives thereof.

FIG. 2 illustrates some of the possible scenarios in which the biounits or biomolecules may be present in the biological entity or the cell. In Panel 1 (on the left) the biounits or biomolecules are associated with a single biological entity or cell, wherein either FIGS. 2—1.a) both biounits or biomolecules are located inside the biological entity or the cell, FIGS. 2—1.b) one biounit or biomolecule is located on the surface of the biological entity or cell and one biounit or biomolecule is located inside the biological entity or cell, or FIGS. 2—1.c) both biounits or biomolecules are located on the surface of the biological entity or cell. In Panel 2 (on the right) the biounits or biomolecules are associated with different biological entities or cells, but are linked through an interaction. In FIGS. 2—2.a) the biounits or biomolecules are located inside the biological entities or cells and an interaction is formed directly between the biological entities or cells. In FIGS. 2—2.b) one biounit or biomolecules located inside one biological entity or cell and the other biounit or biomolecule is located on the surface of the other biological entity or cell, wherein an interaction is formed between a biounit or biomolecule and a biological entity or a cell. In FIGS. 2—2.c) both biounits or biomolecules are located on the surface of the different biological entities or cells, thereby forming an interaction. Scenario (d) is similar to (c), but the interaction between the biounits or biomolecules is enabled through an additional molecule. The term “biomolecule” is interchangeable with “biounit” in this figure and subsequent figures.

FIG. 3 illustrates a possible application of the present invention. B cells are isolated from an individual, such as a human being. Parts of the B cells are immunized with a certain allergen (or alternatively, infected with a pathogen or subjected to a disease state by some other means). The immune repertoire of the B cells is then determined before and after exposure to the allergen and the differences observed are then attributed to the disease.

FIG. 4A and FIG. 4B illustrate the entrapment of the biological entities or cells in an effective range or compartment in accordance with the present invention. The meaning of the symbols and structures used in this FIG. 4 is identical to FIG. 2. Entrapment may, for example, be achieved via an emulsion, such as, for example, a water-in oil emulsion (see FIG. 4A). Entrapment may, for example, also be achieved in the well of a microtiterplate, a picotiterplate, or a sequencing chip (see FIG. 4B). The effective range or compartment is formed by the walls and the lid. The storage may be a bead which comprises binders, e.g., antibodies, which are specific for the biounits, i.e. antigens. Alternatively, the microtiterplate, the picotiterplate, the sequencing chip, or the lid of said vessels may themselves be or serve as storage.

FIG. 5 illustrates an application of the method of the present invention (paired end sequencing). Linker sequences are attached on both ends of a gene of interest. The linker sequences are hybridized to beads comprising tags comprising sequences complementary to both linker sequences. Said tags additionally comprise nucleic acid sequences which serve as starting points for sequencing (x1, x2). The nucleic acid molecules can thereby be sequenced from both ends, leading to substantially longer sequencing reads.

FIG. 6 depicts the coupling of a storage to a binder. Here, an antibody serves as a binder. The antibody comprises at its C-terminus a DNA fragment (sequence A) which is complementary to the DNA fragment of the sequencing bead (sequence A′). The antibody binds to the bead via the complementary nucleic acid sequences A and A′.

FIG. 7 depicts the capture of a biological entity, exemplified via a B cell. The beads loaded with antibodies (output of Example 1) are mixed with B cells, e.g., B cells of an individual infected with a certain pathogen or having a certain disease or disorder. Antibodies with specificity for the B cells bind to the latter, thereby forming a bead-antibody-B cell complex (middle). Antibodies which do not recognize B cells remain unbound (top). Likewise B cells which are not recognized by any antibodies remain unbound (bottom).

FIG. 8 depicts the generation of an effective range or compartment. Due to the size of the cavities each cavity may contain no more than one bead. The following scenarios can occur: (1) a cavity contains a bead with a single cell (left), (2) a cavity contains a bead with two or more cells (middle), (3) a cavity contains one or more cells but no beads (right) and (4) a cavity is empty (not shown).

FIG. 9, FIG. 10 and FIG. 11 depict the binding of the biounits or biomolecules to the storage and amplification. Details are explained in Example 4. Note, that in alternative embodiments sequence A may also be attached to the walls of the cavity or to the lid. This is indicated in the top left corner of FIG. 9.

FIG. 12 depicts the step of detecting and identifying the biounits or biomolecules, exemplified for a nucleic acid. Nucleic acid region C is complementary to nucleic acid sequence C′. Nucleic acid region D is complementary to nucleic acid sequence D′. Details are described in Example 5.

FIG. 13 depicts how sequence data from wells comprising more than one cell can be eliminated from further analysis. Signals from cavities containing beads with two or more cells cannot be readily interpreted since sequencing will deliver mixed signals. Such cavities may be identified by using calibration sequences as described in Example 6. Signal strength in cavities comprising more than one cell differs from the signal strength obtained with the calibration sequence (see bottom) and there will be mixed signals.

FIG. 14A and FIG. 14B depict an embodiment of the present invention in which an emulsion of water in oil is used to generate an effective range. The water droplets comprise the beads with the cells and the PCR mixture (FIG. 14A). FIG. 14B shows the end result after PCR amplification.

FIG. 15 outlines the library-versus-library screening approach. Details are given in Example 8.

### DESCRIPTION OF THE INVENTION

In one aspect the present invention provides a method for the detection of the interaction of two biounits or biomolecules, said method comprising

(a) providing a sample comprising a cell comprising said two interacting biomolecules,

(b) spatially separating said cell in a compartment comprising a moiety which is able to bind derivatives of said biomolecules,

(c) releasing the biomolecules from the cell,

(d) generating derivatives of said biomolecules/units,

(e) allowing the derivatives of said biomolecules to bind to the storage, and

detecting or identifying the derivatives of the biomolecules. In certain aspects said sample comprises more than one cell comprising said two interacting biomolecules. In preferred aspects said two interacting biomolecules are comprised in one cell of said more than one cell. In other aspects said sample comprises at least two cells comprising said two interacting biomolecules. In preferred aspects said two interacting biomolecules are comprised in one cell of said more than one cells. In preferred aspects said two interacting biomolecules are comprised in one cell of said more than one cells.

In one aspect the present invention provides a method for the detection of at least two biounits or biomolecules, said method comprising

(a) providing a sample comprising a biological entity or a cell comprising at least two biounits or biomolecules,

(b) entrapping or spatially separating said biological entity or cell in an effective range or compartment, wherein said effective range or compartment additionally comprises or is itself a storage for said biounits, biomolecules or derivates thereof,

(c) optionally, releasing the biounits from the biological entity,

(d) optionally, generating derivatives of said biounits,

(e) allowing the biounits or their derivatives to bind to the storage, and

(f) detecting or identifying the biounits or their derivatives.

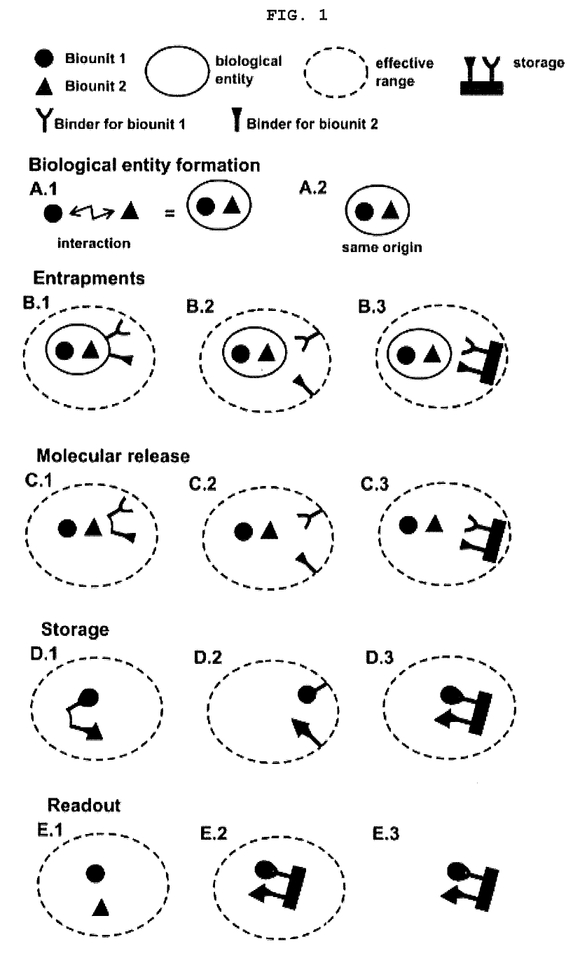

The method is depicted in FIG. 1.

In other aspects the present invention provides a method for the detection of at least two biounits or bimolecules, said method comprising

(a) providing a sample comprising a biological entity or a cell comprising at least two biounits or biomolecules,

(b) entrapping or spatially separating said biological entity or cell in an effective range or compartment, wherein said effective range or compartment comprising a moiety which is able to bind derivatives of said biounits or biomolecules,

(c) releasing the biounits or biomolecules from the biological entity or the cell,

(d) generating derivatives of said biounits or biomolecules,

(e) allowing the derivatives of the biounits or biomolecules to bind to the moiety which is able to bind said derivatives of said biounits or biomolecules, and

(f) detecting or identifying the derivatives of the biounits or biomolecules.

Steps (c) and (d) may be optional. In certain embodiments steps (c) might not be required. This is, for example, the case if two antibodies, both containing a sequencing tag, bind to different epitopes of the same antigen. In such case the antibodies (i.e. the biounits or biomolecules) can be sequenced (i.e. detected or identified) directly, without prior release from the biological entity or the cell. Step (d) may also be optional. The biounits or biomolecules can either be processed directly, or derivatives of the biounits or biomolecules can be generated for further processing. This may be advantageous under certain embodiments, e.g., if the biounits or biomolecules themselves are rather unstable (e.g., mRNA) conversion into more stable formats (e.g., DNA) is preferable.

In one aspect the present invention provides a method for the detection of at least two biounits or biomolecules, said method comprising

(a) providing a sample comprising a biological entity or a cell comprising at least two biounits or biomolecules,

(b) entrapping or spatially separating said biological entity or said cell in an effective range or compartment, wherein said effective range or compartment comprises a moiety which is able to bind said biounits, biomolecules or derivates thereof,

(c) releasing the biounits or biomolecules from the biological entity or the cell,

(d) generating derivatives of said biounits or biomolecules,

(e) allowing the biounits, biomolecules or derivatives thereof to bind to the moiety which is able to bind said biounits, biomolecules or derivates thereof, and

(f) detecting or identifying the biounits, biomolecules or their derivatives.

In one aspect the present invention provides a method for the detection the interaction of two biounits or biomolecules, said method comprising

(a) providing a sample comprising a biological entity or a cell comprising said two interacting biounits or biomolecules,

(b) entrapping or spatially separating said biological entity or said cell in an effective range or compartment, wherein said effective range or compartment comprises a moiety which is able to bind said biounits, biomolecules or derivates thereof,

(c) releasing the biounits or biomolecules from the biological entity or the cell,

(d) generating derivatives of said biounits or biomolecules,

(e) allowing the biounits, biomolecules or derivatives thereof to bind to the moiety which is able to bind said biounits, biomolecules or derivates thereof, and

(f) detecting or identifying the biounits, biomolecules or their derivatives.

In an alternative aspect the present invention provides a method for the detection of at least two biounits or biomolecules, said method comprising

(a) providing a sample comprising a biological entity or a cell comprising at least two biounits or biomolecules,

(b) entrapping or spatially separating said biological entity or cell in an effective range or a compartment, wherein said effective range or compartment comprises a moiety which is able to bind said biounits, biomolecules or derivates thereof,

(c) generating derivatives of said biounits or biomolecules,

(d) allowing the derivatives of the biounits or biomolecules to bind to the moiety which is able to bind said biounits, biomolecules or derivates thereof, and

(e) detecting or identifying the biounits, biomolecules or their derivatives.

In an alternative aspect the present invention provides a method for the detection of the interaction of two biounits or biomolecules, said method comprising

(a) providing a sample comprising a biological entity or a cell comprising said two interacting biounits or biomolecules,

(b) entrapping or spatially separating said biological entity or cell in an effective range or a compartment, wherein said effective range or compartment comprises a moiety which is able to bind said biounits, biomolecules or derivates thereof,

(c) generating derivatives of said biounits or biomolecules,

(d) allowing the derivatives of the biounits or biomolecules to bind to the moiety which is able to bind said biounits, biomolecules or derivates thereof, and

(e) detecting or identifying the biounits, biomolecules or their derivatives.

In yet alternative aspect the present invention provides a method for the detection of at least two biounits or biomolecules, said method comprising

(a) providing a sample comprising a biological entity or a cell comprising at least two biounit or biomolecules,

(b) entrapping or spatially separating said biological entity or cell in an effective range or compartment, wherein said effective range or compartment comprises a moiety which is able to bind said biounits or biomolecules, releasing the biounits or biomolecules from the biological entity or the cell,

(c) allowing the biounits or biomolecules to bind to the moiety which is able to bind said biounits or biomolecules, and

(d) detecting or identifying the biounits or biomolecules.

In yet alternative aspect the present invention provides a method for the detection the interaction of two biounits or biomolecules, said method comprising

(a) providing a sample comprising a biological entity or a cell comprising said two interacting biounit or biomolecules,

(b) entrapping or spatially separating said biological entity or cell in an effective range or compartment, wherein said effective range or compartment comprises a moiety which is able to bind said biounits or biomolecules,

(c) releasing the biounits or biomolecules from the biological entity or the cell,

(d) allowing the biounits or biomolecules to bind to the moiety which is able to bind said biounits or biomolecules, and

(e) detecting or identifying the biounits or biomolecules.

In yet alternative aspect the present invention provides a method for the detection of at least two biounits or biomolecules, said method comprising

(a) providing a sample comprising a biological entity or cell comprising at least two biounits or biomolecules,

(b) entrapping or spatially separating said biological entity or cell in an effective range or compartment, wherein said effective range or compartment comprises a moiety which is able to bind said biounits or biomolecules,

(c) allowing the biounits to bind to the moiety which is able to bind said biounits or biomolecules, and

(d) detecting or identifying the biounits or biomolecules.

In yet alternative aspect the present invention provides a method for the detection the interaction of two biounits or biomolecules, said method comprising

(a) providing a sample comprising a biological entity or cell comprising said two interacting biounits or biomolecules,

(b) entrapping or spatially separating said biological entity or cell in an effective range or compartment, wherein said effective range or compartment comprises a moiety which is able to bind said biounits or biomolecules,

(c) allowing the biounits to bind to the moiety which is able to bind said biounits or biomolecules, and

(d) detecting or identifying the biounits or biomolecules.

The at least two biounits or biomolecules of the present invention may be present in one biological entity. Alternatively, the at least two biounits or biomolecules of the present invention may be present in one cell. In certain embodiments both biounits or biomolecules may be located inside the biological entity or the cell. In alternative embodiments both biounits or biomolecules may be located on or outside the biological entity or cell. In yet alternative embodiments one biounit or biomolecules may be located inside the biological entity or cell and the other biounit or biomolecule may be located on or outside the biological entity or cell.

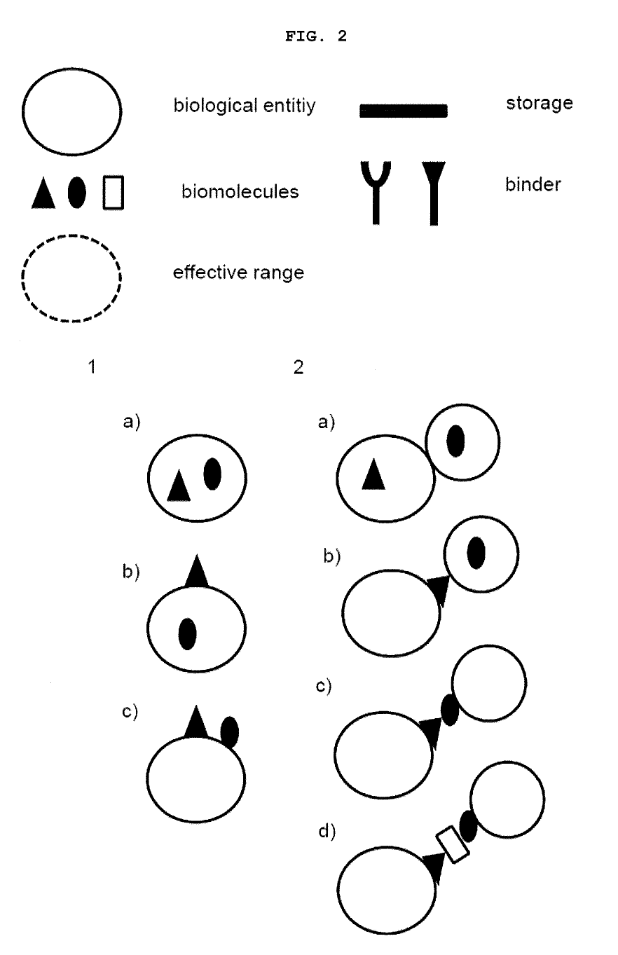

The at least two biounits or biomolecules may also be present in two biological entities or cells which interact with each other in any way or form. In certain embodiments a first biounit or biomolecule is located inside a first biological entity or cell and a second biounit or biomolecule is located inside a second biological entity or cell. In other embodiments a first biounit or biomolecule is located on or outside a first biological entity or cell and a second biounit or biomolecule is located inside a second biological entity or cell. In yet other embodiments a first biounit or biomolecules is located on or outside a first biological entity or cell and a second biounit or biomolecule is located on or outside a second biological entity or cell. In yet other embodiments a first biounit or biomolecule is located on or outside a first biological entity or cell and a second biounit or biomolecule is located on or outside a second biological entity or cell, and the two biounits or biomolecules are interacting indirectly via a third molecule, e.g., a biounit or biomolecule, which binds to both, the first biounit or biomolecule and the second biounit or biomolecule. FIG. 2 illustrates some of the possible scenarios.

Methods in the prior art that are used in these technology areas include the yeast two-hybrid system, the yeast three-hybrid system, the SIP technology (self-infective phage), PCA assays (protein-fragment complementation assays) and the split-ubiquitin system. All these assays and systems suffer from a high background noise of the read out signal, leading to an unsatisfactory signal-to-background ratio. Mostly this is due to the unspecific interactions that occur in these systems. Avoiding these problems by quantifying the readout signals, e.g., by measuring color intensity or colony size, is only partly successful and still troublesome and error-prone. The present invention provides an elegant solution to these shortcomings. By analyzing thousands or even millions of interactions or events, the problem of receiving a statistically significant read-out signal is solved by increasing the number of interactions or events that are analyzed. The present invention provides a high throughput method capable of analyzing a large number of read out signals. This can for example be achieved at the genotype level, in cases where the respective phenotypic read out signals suffer from a low signal-to-background ratio.

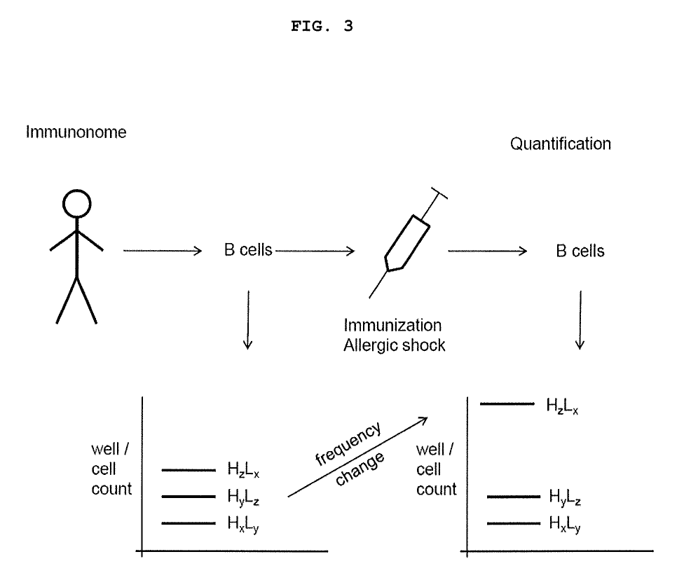

The present invention can also be used in library-versus-library applications, e.g., the screening for interactions between the members of one library with members of a second library. As one example, the immune response of an organism, such as the immune repertoire of a human being in response to an infection or a disease, could be measured. The complex analysis of the entire immune response in a statistically significant and satisfactory manner has only become possible with the methods described in the present invention. A respective experimental approach is depicted in FIG. 3.

In certain aspects said biounits are selected from the group consisting of the sub-classes polypeptides, proteins, peptides, nucleic acids, carbohydrates, fatty acids, small molecules, cell organelles, cells, tissues, or derivatives, parts or combinations of any of the foregoing. In certain aspects all biounits are from the same subclass. In certain aspects the biounits are from different subclasses. In certain aspects said subclass is the subclass of polypeptides or the subclass of nucleic acids. In certain aspects said subclass is the subclass of polypeptides.

In particular aspects said biounits are biomolecules. In certain preferred aspects said biounits or biomolecules are proteins, polypeptides or peptides. In alternative aspects said biomolecules are nucleic acids, such as DNA or RNA. Said nucleic acids may by single stranded or double stranded. It will be understood that DNA molecules can be synthesized from RNA or DNA templates. Likewise, RNA molecules can be synthesized from RNA or DNA templates as well.

In certain aspects said biounits or biomolecules are part of a multimeric protein or enzyme. In certain aspects said biounits or biomolecules are polypeptides which are part of a multimeric protein or enzyme. In certain aspects said biounits or biomolecules are nucleic acids which encode for part of a multimeric protein or enzyme. In certain aspect said multimeric protein is an immunoglobulin, or a functional fragment thereof. In certain aspect said biounits or biomolecules are genes encoding the variable heavy and the variable light chain of an immunoglobulin or a functional fragment thereof.

In certain aspects the sample used in the present invention is a sample derived from blood, bone marrow, a tumor, a single cellular organism, a prokaryote or a body fluid. In certain aspects said sample is a sample from a patient, wherein said patient is a healthy patient, an immunized patient, an infected patient or a patient with a disease or disorder.

In certain aspects the biological entity used in the present invention is a single cell. In certain aspects said single cell is a single B cell.

In certain aspects the biological entity is a cell interacting with another cell, a virus, a bacterium, a molecule or a biomolecule, or derivatives, fragments or composites of any of the foregoing. In certain aspects the biological entity is a cell and a virus infecting said cell. In certain aspects the biological entity is a cell and a bacterium infecting said cell. In certain aspects the biological entity is a cell and another cell. Said cell may communicate with each other in terms of a donor and an acceptor, in terms of an effector and an effected entity, or in terms of an inhibitor and an inhibited cell.

In certain aspects the biological entity comprises a mixture of two chemical and/or biological libraries (such as, for example, a phage library or a ribosome display library), wherein at least one member of the first library interacts with or binds to a member of the second library. In certain aspects each member of the first library comprises a tag which is specific for the first library, and the second library comprises a tag which is specific for the second library.

In certain aspects the biological entity comprises a molecule which interacts with or binds to at least two members of at least one chemical and/or biological library (such as for example a phage library or a ribosome display library). In certain aspects each member of a library comprises a tag which is specific for said library.

In alternative aspects the provides a method for the detection of at least two biounits or biomolecules, said method comprising

(a) providing a sample comprising a biological entity or cell comprising at least two biounits or biomolecules,

(b) entrapping or spatially separating said at least two biounits or biomolecules and a moiety which is able to bind said biounits or biomolecules in an effective range or compartment,

(c) optionally, releasing the biounits or biomolecules from the biological entity or cell,

(d) allowing the biounits or biomolecules to bind to the moiety which is able to bind said biounits or biomolecules, and

(e) detecting or identifying the biounits or biomolecules on the moiety which is able to bind said biounits or biomolecules.

In alternative aspects the provides a method for the detection of the interaction of two biounits or biomolecules, said method comprising

(a) providing a sample comprising a biological entity or cell comprising said two interacting biounits or biomolecules,

(b) entrapping or spatially separating said at least two biounits or biomolecules and a moiety which is able to bind said biounits or biomolecules in an effective range or compartment,

(c) optionally, releasing the biounits or biomolecules from the biological entity or cell,

(d) allowing the biounits or biomolecules to bind to the moiety which is able to bind said biounits or biomolecules, and

(e) detecting or identifying the biounits or biomolecules on the moiety which is able to bind said biounits or biomolecules.

In alternative aspects the provides a method for the detection of at least two biounits or biomolecules, said method comprising

(a) providing a sample comprising a biological entity or cell comprising at least two biounits or biomolecules,

(b) entrapping or spatially separating said at least two biounits or biomolecules in an effective range or compartment, wherein said effective range or compartment is able to bind said biounits or biomolecules,

(c) optionally, releasing the biounits or biomolecules from the biological entity or cell,

(d) allowing the biounits or biomolecules to bind to the effective range or compartment, and

(e) detecting or identifying the biounits or biomolecules on the effective range or compartment.

In alternative aspects the provides a method for the detection of the interaction of two biounits or biomolecules, said method comprising

(a) providing a sample comprising a biological entity or cell comprising said two interacting biounits or biomolecules,

(b) entrapping or spatially separating said at least two biounits or biomolecules in an effective range or compartment, wherein said effective range or compartment is able to bind said biounits or biomolecules,

(c) optionally, releasing the biounits or biomolecules from the biological entity or cell,

(d) allowing the biounits or biomolecules to bind to the effective range or compartment, and

(e) detecting or identifying the biounits or biomolecules on the effective range or compartment.

In one step the method of the present invention comprises the entrapment of at least one biological entity or cell comprising at least two biounits or biomolecules and a storage for said biounits or biomolecules in an effective range or compartment. Aforementioned storage is a moiety which is able to bind said biounits or biomolecules or derivatives thereof. In certain embodiments said storage is a moiety which is able to bind said biounits or biomolecules. In other embodiments said storage is a moiety which is able to bind derivatives of said biounits or biomolecules.

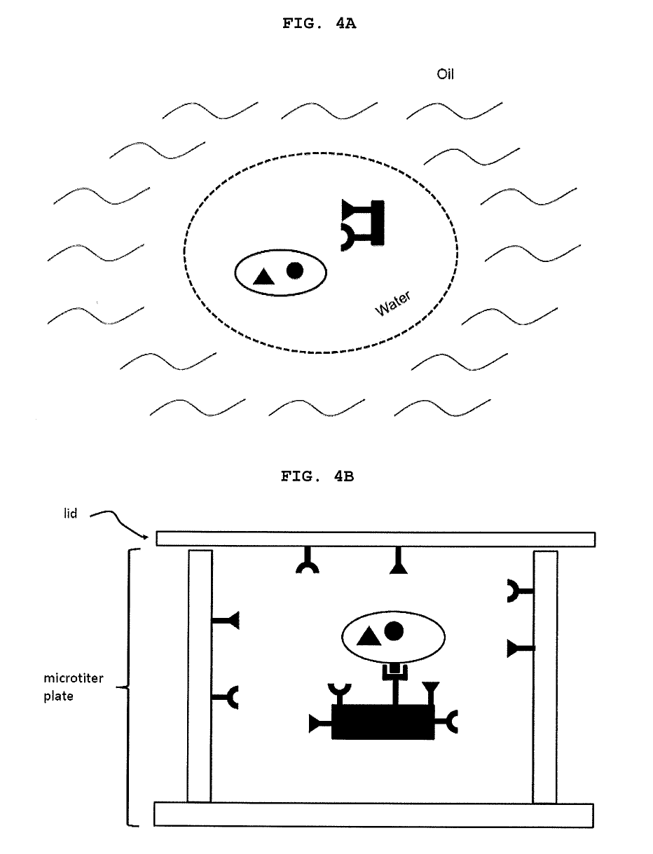

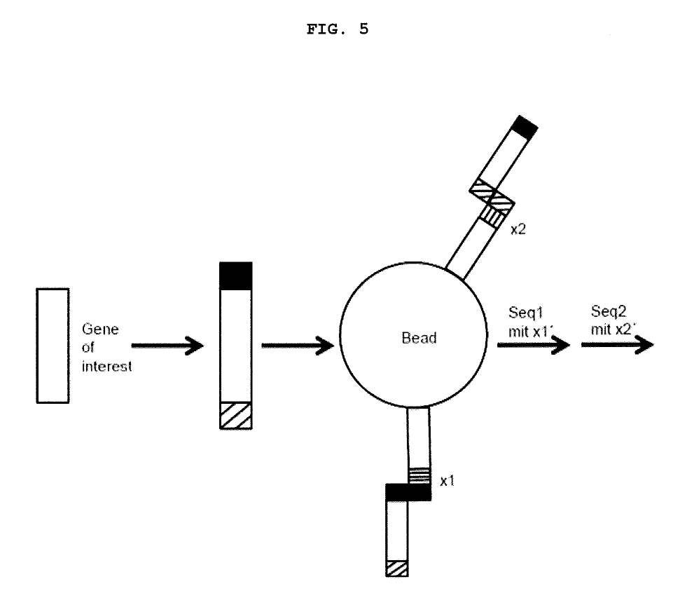

The entrapment or spatially separation of said biological entities or cells, biounits or biomolecules, and the storage may be achieved in any way or form that enables the subsequent detection of the biounits or biomolecules entrapped or spatially separated. In certain embodiments this can be achieved via an emulsion, such as for example an water-in oil emulsion (see FIG. 4A). In such embodiments the biological entity may be a cell, the biounits or biomolecules may be DNA sequences (e.g., the variable heavy chain and the variable light chain of an antibody), and the storage may be primers (e.g., one primer binding to the variable heavy chain of an antibody and another primer binding to the variable light chain of the same antibody). As an alternative to a water-in-oil emulsion, an oil-in-water emulsion may be used, thereby forming micelles which serve as an effective range or compartment. In other embodiments the entrapment or spatial separation of said biological entities or cells, biounits or biomolecules, and the storage may be achieved in the wells of a microtiterplate, a picotiterplate, or a sequencing chip (see FIG. 4B). The effective range or compartment is formed by the walls and the lid. In such embodiments the biological entity may be a cell, the biounits or biomolecules may be antigens and the storage may be a bead which comprises binders, e.g., antibodies, which are specific for the biounits, i.e. antigens. Alternatively, the microtiterplate, the picotiterplate, the sequencing chip, or the lid of said vessels may themselves be or serve as storage. Again, the storage comprises at least two binders, e.g., primers.

In certain aspects of the present invention said effective range or compartment is formed by a cavity, a well, an emulsion, a phase-boundary-system, a hydrophobic spot, a particle, a solid-phase particle, physical forces or chemical cross-linking. In certain aspects of the present invention said phase boundaries are realized by a phase separation between water and gas like water droplets in air or water and a liquid like water droplets in oil or water and a solid phase like water droplets in a microtiterplate. In certain aspects of the present invention said cavity or said well is on a microtiterplate, a picotiterplate or a microstructured substrate. In certain aspects of the present invention said effective range is formed chemical cross-linking, wherein said chemical cross-linking is cross-linking with formaldehyde.

In certain aspects of the present invention, said biounits or biomolecules and said storage for said biounits, biomolecules or derivatives thereof are entrapped or spatially separated in an effective range or compartment by limited dilution or by sorting by any means, such as cell sorting.

In certain aspects of the present invention, the effective range or compartment is formed by an emulsion, wherein said emulsion is a water-in-oil or an oil-in-water emulsion.

In certain aspects of the present invention, the effective range or compartment is formed by a particle or a solid-phase particle, wherein said particle consists of silica, glass, agarose, a polymer, a metal oxide or a composite thereof.

In certain aspects of the present invention, the effective range or compartment is formed by physical forces, wherein said physical forces are electrostatic forces, electrodynamic forces, dielectrophoretic forces, electromagnetic forces, magnetic, optical, temperature or density effects. In certain aspects of the present invention, the effective range or compartment is generated by an optical tweezer.

In certain aspects of the present invention, the effective range or compartment is formed by a nebulizer.

In certain aspects of the present invention, said storage or said moiety which is able to bind the biounits, biomolecules or derivatives thereof is a bead, an area on the surface of a glass slide, a well of a microtiterplate or picotiterplate, an electric or magnetic field, field gradient or field cage, or an area on the lid or a separable surface of any of the foregoing.

In certain aspects of the present invention, the release of the biounits or biomolecules from the biological entity or cell is performed by a change of the chemical or physical conditions. In certain aspects the change of chemical conditions is a pH change, a change of salt concentrations, the addition of an enzyme or the addition of lytic agents. In certain aspects the change of physical conditions is heating, freezing, application of electric, magnetic or dielectric fields, sheer or centrifugal forces, mechanical deformation, relaxation, ultrasonic or any physical disruptive effect. In certain aspects the change of physical conditions is heating, e.g., heating to more than 90° C. In certain aspects said change of the physical condition is effected in a time dependent manned, such as dissolving of a particle in a solution, the dissolving of a protective shell around the biounit or biomolecule or the induction by an enzyme.

In another step the method of the present invention comprises allowing the biounits or biomolecules to bind to the storage or the moiety which is able to bind the biounits, biomolecules or derivatives thereof. This step includes incubating the biounits or biomolecules for a time and in a manner sufficient to allow binding of the biounits or biomolecules to said storage or said moiety. By doing so, the biounits or biomolecules are captured by the storage the moiety which is able to bind the biounits, biomolecules or derivatives thereof. The binding of the biounits or biomolecules may be directly or indirectly via a derivative of the biounit or the biomolecule. Direct binding may for example be achieved via PCR or a direct reaction. Indirect binding may be achieved via the generation of a derivative of the biounit or the biomolecule and binding of said derivative to the storage or the moiety which is able to bind such derivative. One example is the generation of a cDNA template from RNA and binding of the cDNA to the storage, e.g., a primer.

Therefore, in certain aspects of the present invention, step (d) includes an amplification reaction which leads to the generation of replicates or derivatives of said biounits or biomolecules. In certain aspects of the present invention, said amplification reaction is a PCR or a RT-PCR, and wherein during said PCR or RT-PCR a [first] tag is added which enables said replicates or derivatives to bind to the storage or moiety which is able to bind such derivative. In certain aspects of the present invention, during said PCR or RT-PCR a second tag is added which enables subsequent sequencing of the PCR or RT-PCR product. In certain aspects of the present invention the PCR reaction is an emulsion PCR reaction. In alternative aspects the RT-PCR reaction is an emulsion RT-PCR. In certain aspects of the present invention the two nucleic acid molecules to be detected are amplified in step (d) of the present method.

In another step the method of the present invention comprises detecting the biounits or biomolecules on the storage or on the moiety which is able to bind the biounits or biomolecules. This can be achieved via any suitable detection method known for the biounits or bimolecules to be detected, and the method to be used mainly depends on the nature of the biounit or the biomolecule itself. Examples are (at least) two immunoassays which are performed in parallel or subsequently, parallel staining with at least two dyes and parallel sequencing.

In certain aspects of the present invention, the detection of the biounits or biomolecules is performed by DNA sequencing. In certain aspects of the present invention, said DNA sequencing is performed by sequencing the PCR or RT-PCR products sequentially or in parallel. In certain aspects of the present invention, said DNA sequencing is performed by sequencing the PCR or RT-PCR products on the storage or on copies of the storage. In certain aspects of the present invention the biounits or biomolecules to be detected are nucleic acid molecules and different start primers are used for sequencing and identification of the nucleic acid molecules. In certain aspects of the present invention the sequencing reaction is performed by emulsion PCR, preferably directly on the bead. In certain aspects of the present invention two sequencing reactions are performed subsequently, e.g., a first sequencing reaction utilizing a first sequencing primer and a second sequencing reaction utilizing a second sequencing primer. In certain embodiments the first nucleic acid molecule encodes for the heavy chain of an immunoglobulin, an antibody or a fragment thereof and the second nucleic acid molecule encodes for the light chain of an immunoglobulin, an antibody or a fragment thereof.

Certain technological variations are possible if the detection step of the methods of the present invention is performed via sequencing. For example, the primers for the second sequencing reaction could be linked to an enzymatically cleavable protection group. This would ensure that the second nucleic acid molecule can only be sequenced after cleavage of the protection group. This will decrease sequencing errors. Alternatively, the second sequencing primer may be added subsequently, i.e. after the first sequencing reaction was performed. Also, certain nucleic acid motifs may be used and attached to the nucleic acid molecules to be detected. These nucleic acid motifs are used in a kind of “ZIP code” to identify or mark the nucleic acid molecules.

Exemplary sequencing systems that may be used in the methods of the present invention include the GS FLX 454 system (Roche) and the HiSeq2000 system (Illumina).

In certain aspects of the present invention, the sample comprises at least 10³, at least 10⁶, at least 10⁹ or at least 10¹² biological entities or cells. In certain aspects of the present invention in each of said biological entities or cells at least two, at least three, at least five, at least 10, at least twenty, at least fifty, at least one hundred, at least five hundred, or at least one thousand biounits or biomolecules are detected. In certain aspects of the present invention, the sample comprises at least 10³, at least 10⁶, at least 10⁹ or at least 10¹² biological entities or cells, and at least two, at least three, at least five, at least 10, at least twenty, at least fifty, at least one hundred, at least five hundred, or at least one thousand biounits or biomolecules are detected wherein in each of said biological entities or cells.

In certain aspects of the present invention, the correlation of the presence of said at least two biounits or biomolecules within said biological entities or cells, or the interaction of said two biounits or biomolecules is statistically analyzed or determined. Appropriate statistical analysis tools are known to the person skilled in the art. Non-limiting examples of appropriate statistical tools include the analysis or determination of the covariance or the correlation coefficient according to Bravais-Pearson or according to Spearman.

In certain aspects of the present invention said biounits or bimolecules are nucleic acids that bind by hybridization to a particle, and said particle is used for sequencing in step (e).

In certain aspects of the present invention said biounits or biomolecules are polypeptides, peptides or proteins that bind directly to the surface of the particle, and wherein said biounit or biomolecule on said particle is detected via an immunoassay in step (e).

In certain embodiments the present invention provides a method for the detection of at least two biounits or biomolecules in a biological entity or a cell, said method comprising

(a) entrapping or spatially separating at least one biological entity or cell comprising at least two biounits or biomolecules and a storage for said biounits or biomolecules in an effective range or compartment,

(b) optional releasing the biounits or biomolecules from the biological entity or cell,

(c) allowing the biounits or biomolecules to bind to the storage, and

(d) detecting or identifying the biounits or biomolecules on the storage.

In alternative embodiments the present invention provides a method for the detection of the interaction of two biounits or biomolecules in a biological entity or a cell, said method comprising

(a) entrapping or spatially separating at least one biological entity or cell comprising at least two biounits or biomolecules and a storage for said biounits or biomolecules in an effective range or compartment,

(b) optional releasing the biounits or biomolecules from the biological entity or cell,

(c) allowing the biounits or biomolecules to bind to the storage, and

(d) detecting or identifying the biounits or biomolecules on the storage.

In other embodiments the present invention provides a method for the detection of at least two biounits or biomolecules in a biological entity or a cell, said method comprising

(a) entrapping or spatially separating at least one biological entity or cell comprising at least two biounits or biomolecules in an effective range or compartment, wherein said effective range or compartment is also a storage,

(b) optional releasing the biounits or biomolecules from the biological entity or cell,

(c) allowing the biounits or biomolecules to bind to the storage, and

(d) detecting or identifying the biounits or biomolecules on the storage.

In other embodiments the present invention provides a method for the detection of the interaction of two biounits or biomolecules in a biological entity or a cell, said method comprising

(a) entrapping or spatially separating at least one biological entity or cell comprising at least two biounits or biomolecules in an effective range or compartment, wherein said effective range or compartment is also a storage,

(b) optional releasing the biounits or biomolecules from the biological entity or cell,

(c) allowing the biounits or biomolecules to bind to the storage, and

(d) detecting or identifying the biounits or biomolecules on the storage.

Said at least two biounits or biomolecules may or may not interact with each other. Said biounits or biomolecules may only be present in the respective biological entity, e.g., a cell, but might not directly interact with each other. They may however be linked via a common event, e.g., a stimulus which triggers for example the expression of certain genes and the subsequent production of the respective polypeptides. Said biounits or biomolecules might also be biounits or biomolecules that interact with each other. Examples are two polypeptides that bind to each other or form a complex with each other or through a third molecule. Another example are the subunits of multimeric proteins, for example the variable heavy chain and the variable light chain of an immunoglobulin, such as an antibody.

If the at least two biounits or biomolecules interact with each other, then the method provided by the present invention may be rephrased as a method for the detection of the interaction of at least two biounits or biomolecules in a biological entity or a cell, said method comprising

(a) entrapping or spatially separating at least one biological entity or cell comprising at least two interacting biounits or biomolecules and a storage for said biounits or biomolecules in an effective range or compartment,

(b) releasing the biounits or biomolecules from the biological entity or the cell,

(c) allowing the biounits or biomolecules to bind to the storage, and

(d) detecting or identifying the biounits or biomolecules on the storage.

Alternatively, the present invention provides a method for the detection of the interaction of at least two biounits or biomolecules in a biological entity or a cell, said method comprising

(a) entrapping or spatially separating at least one biological entity or cell comprising at least two interacting biounits or biomolecules and a moiety which is able to bind the biounits, biomolecules or derivatives thereof, in an effective range or compartment,

(b) releasing the biounits or biomolecules from the biological entity or the cell,

(c) allowing the biounits or biomolecules to bind to the moiety which is able to bind the biounits, biomolecules or derivatives thereof, and

(d) detecting or identifying the biounits or biomolecules on the moiety which is able to bind the biounits, biomolecules or derivatives thereof.

In alternative embodiments, if at least two biounits or biomolecules interact with each other, then the method provided by the present invention may be rephrased as a method for the detection of the interaction of at least two biounits or biomolecules in a biological entity or a cell, said method comprising

(a) entrapping or spatially separating at least one biological entity or cell comprising at least two interacting biounits or biomolecules in an effective range or compartment, wherein said effective range or compartment is also a storage

(b) releasing the biounits or biomolecules from the biological entity or the cell,

(c) allowing the biounits or biomolecules to bind to the moiety which is able to bind the biounits, biomolecules or derivatives thereof, and

(d) detecting or identifying the biounits or biomolecules on the moiety which is able to bind the biounits, biomolecules or derivatives thereof.

This method can be adapted for the screening of interactions between the biounits or biomolecules comprised in a first library and the biounits or biomolecules comprised in a second library. Principally it is for example possible to identify all protein-protein interactions in a given organism or cell.

In certain embodiments of the present invention display technologies are used to present the biounits or biomolecules of the present invention, in particular if the biounits or biomolecules are comprised in a respective library. Phage display technologies and ribosome display technologies are particularly useful for the display of proteinaceous biounits or biomolecules, such as proteins, polypeptides or peptides.

As described herein above the present invention can be used to screen a first library of biounits or biomolecules against a second library of biounits or biomolecules. This leads to the identification of all interaction between the biounits or biomolecules of the first library with the biounits or biomolecules of the second library.

Another application of such a library-versus-library approach is the identification of two biounits or biomolecules that both bind to a common target protein. The biounits or biomolecules so identified will bind to the same target molecule, but at different epitopes of said target molecule. Respective pairs of such biounits or biomolecules are for example useful in ELISA assays. Experimentally, a target molecule comprising a tag is incubated with two libraries, e.g., phage display libraries, at conditions that allow members of said libraries to bind to said target molecule. The complex comprising the target molecule and the phage display members of said library binding to said target are isolated via the tag on said target molecule which has affinity to a bead. Further processing as described in the present invention will lead to the identification of two biounits or biomolecules which bind to the target molecule simultaneously, i.e. at the same time and not interfering with each other. The high redundancy and throughput, which is only possible with the present method, solves the problem of the identification of unspecific or sticky binders which are very often identified in similar experiments of the prior art.

Another application of the library-versus-library approach is the identification of biounits or biomolecules which interact or bind to only one isoenzymatic form of an enzyme. For example, a first library of biounits or biomolecules is incubated with a first isoform of a given enzyme. Then a second library of biounits or biomolecules is incubated with a second isoform of said enzyme, wherein the members of said second library comprise a tag. The two libraries are then mixed in a manner so that the first library is present in excess of the second library. Next, beads with affinity for the tag are added to isolate phages which bind only to the second isoform, but not to the first isoform.

Another application of a library-versus-library approach is the screening of an antibody library against a mixed population of bacteria, e.g., bacteria of different species or subspecies. The population of bacteria is mixed with an antibody library, wherein each antibody comprises a DNA tag. The antibody-bacterium complexes are isolated via the DNA tag. The isolated bacterium can be identified by way of sequencing its 16S rRNA or any other sequence suitable for the genetic identification of bacteria. This will lead to the identification of antibodies specific for certain bacteria isolated from a mixed population, and at the same time information about the species and the subspecies of the bacterium can be collected.

Other uses of the method are in yeast-two-hybrid and yeast-three-hybrid applications. A big advantage lies in the highly parallel fashion of the technology of the present invention which enables statistically meaningful analysis of the data. Other suitable technologies that may also be used in context with the methods of the present invention are the SIP technology (self-infective phage), PCA (protein complementation assay) or other technologies suitable for the identification of protein-protein, or protein-ligand interactions, e.g., pathogen-host, host-symbiont or host-parasite interactions.

The present invention can also be used to identify gene that are expressed simultaneously, for example in response to a certain stimulus or a change of certain conditions in the environment. Such analysis may be performed qualitatively or quantitatively. Quantitative analysis is possible through the high parallel fashion in which the present invention can be employed.

Cells, or other biological entities, to be analyzed may be cancer cells, cells infected with a certain pathogen or cells treated with certain pharmaceutical agents. Events that may be detected with the methods of the present invention include the co-expression of genes or polypeptides, the detection of the expression of certain genes in response to, e.g., an infection, the resistance pattern of cells, or the differential expression of cells in different tissue.

The present invention can also be used to identify and characterize the immunonome of a cell, such as a B cell, a tissue or an organism. In particular it is possible to identify which variable heavy chain of an immunoglobulin is paired with which variable light chain. This information can be used to characterize the immune repertoire of an organism. Such information can furthermore be used to compare the immune repertoire of a healthy patient with the immune repertoire of a sick or infected patient (see FIG. 2).

The present invention can also be used to identify, dissect and characterize pathways. The quantitative sequencing of two or more genes of a given pathway may be used as a standard for the evaluation of the mode of action of, for example, drug libraries. The effect of the drugs can be measured directly by quantification of the transcripts. No indirect and inaccurate quantification via reporter genes is necessary.

The present invention can also be used to overcome difficulties and pitfalls associated with nucleic acid sequencing. Obviously, the present invention provides the advantage of high throughput and high parallel sequencing. This enables the generation of highly complex sequence populations, such as entire genomes, immunonomes, or the analysis of SNPs (single nucleotide polymorphisms). Also, as already discussed in the present invention, it is possible to compare different populations of nucleic acid molecule, such as pre-treatment versus post-treatment, healthy versus sick, or any other pool of nucleic acid molecules that need to be compared.

The present invention also overcomes the difficulties of the limited reading length capacity of standard sequencing technologies. By using respective hybridization primers, or alternatively known sequence stretches of the gene to be sequenced, it is possible to sequence a respective nucleic acid molecule from both ends, thereby essentially duplicating the common reading length (see FIG. 5).

Another possibility of the present invention is the identification and characterization of enzymatic chains. Cells are transfected with two different plasmids: plasmid #1 encodes for an enzyme #1 which comprises a tag #1 and plasmid #2 encodes for an enzyme #2 which comprises a tag #2. Cells are entrapped or spatially separated in an effective range or compartment and lysed. Beads with specificity for both tags, i.e. tag #1 and tag #2, are then used to capture the respective enzymes. The beads are then tested for enzymatic activity. For example, a substrate for enzyme #1 is added and the substrate is converted into a respective first product by enzyme #1. If the first product is not directly detectable, it now can be further converted into another, detectable, product by enzyme #2. I.e., the product of the first enzymatic reaction is the edict for the second enzymatic reaction, and the final product is a detectable, e.g., fluorescent, substance. Variations of this method can also be used to identify natural or synthetic co-enzymes, to detect isoenzymes and/or quantify the ratio of isoenzymes within a cell, or to identify co-factors, co-enzymes or inhibitors, such as allosteric inhibitors.

Other possibilities of the present invention include examinations with respect to the MHC, such as determination of the MHC isotypes of a sample or a patient, or MHC screenings with the aim to identify MHC-reactive antibodies with certain properties, e.g., MHC-reactive antibodies which suppress or enhance the T cell response.

Yet other applications of the present invention relate to the determination and characterization of the binding motives of nucleic acids, such as DNA or RNA, of promoters, of activators, of silencers, or of any other elements, such as regulatory elements. This includes the screening for binding motives for siRNA-based interventions, such as gene therapy. Other related uses relate to aptamer screening.

Yet other applications of the present invention relate to the identification of target molecules, for example target proteins, of a given molecule of interest. It is also possible to study the effect of molecules on a known interaction, for example the effect a certain compound has on a known interaction between a first and a second polypeptide in a patient.

Yet other applications of the present invention relate to the analysis of germ cells, such as an ovocyte or a sperm cell. This includes the identification of one or more genes or one or more alleles, for example for the determination of the gender of an embryo or the determination with respect to genetic predispositions.

Yet other applications of the present invention relate, generally, to the identification of inhibitors, inducers, mutations or any other changes that cause or relate to a certain effect that is observed or that is aimed to be observed.

The methods of the present invention can be incorporated in assays of various types, for example immunoassays. Therefore, in certain aspects the present invention provides assays, e.g., immunoassays, which incorporate or utilize any of the methods of the present invention.

In certain aspects the present invention provides a device for performing a method or an immunoassay of the present invention.

In certain aspects the present invention provides a kit comprising a device and instruction to perform a method or an immunoassay of the present invention.

In yet other aspects the methods of the present invention provide certain information, products or information about certain products which, on their own, might be used for various subsequent methods.

The methods provided with the present invention can be adapted to numerous applications.

In certain embodiments the present invention provides a method for the detection of two or more biomolecules in a cell. Said cells are separated in order to preserve the information that the biomolecules to be detected derive from the same cell. In certain aspects it is preferable to generate derivatives of said biomolecules. In certain aspects of the present invention the biomolecules detected in said cell interact with each other in said cell. An example for the latter aspect is the detection of the subunits of multimeric enzymes, e.g., the heave chain polypeptide and the light chain polypeptide of an immunoglobulin or a fragment thereof.

Any biomolecule can be detected in this methods. Preferred biomolecules are polypeptidic biomolecules, such as peptides, polypeptides and proteins, and nucleic acids, such as ribonucleic acid or deoxyribonucleic acids. Typical derivatives in accordance with the present invention are cDNA molecules which are prepared by reverse transcribin RNA molecules, e.g., mRNA. This not only leads to more stable molecules that can be detected, but at the same time the molecules can be amplified, e.g., by PCR, in order to increase the number of the copies of the biomolecules or the derivatives to be detected, thereby increasing sensitivity of the respective method. This is further facilitated by the moiety which is able to bind the biomolecules or derivatives thereof thereby retaining the biomolecules or derivatives in the compartment for subsequent analysis and detection.

Examples that fall under this aspect of the invention include the detection of the nucleic acids encoding the heavy chain and the light chain of an antibody in, e.g., a B cell. Mature B cells produce one antibody species and thereby produce large quantities of the respective mRNAs. Detection of the mRNAs encoding the heavy chain and the light chains in a large number of B cells (e.g., a large representative population of B cells of a patient with a certain type of disease or disorder) thereby not only provides the information which heavy chain mRNAs and which light chain mRNAs are produced by such cell, but additionally provides the valuable information which heavy chain of the antibody is paired with which light chain in each individual B cell. This provides a rational to directly de novo synthesize and test the respective antibodies for efficacy, e.g., therapeutic efficacy.

Likewise any other mRNA molecules can be detected by the same approach as well. Due to the high throughput that can be achieved with the method of the present invention, a statistical analysis can be employed that makes it possible to link the appearance of certain mRNAs with certain diseases, disorders or any other condition that are investigated. The method is therefore suited for the identification of biomarkers for such diseases, disorders or conditions.

If such biomarkers are already known, the present invention can be used to identify the occurrence or presence of such biomarkers in any given sample, e.g., a sample obtained from a patient, such as sputum, saliva, liquor, blood or any other body fluid. The high throughput of the method also makes it possible to quantify the presence of certain biomarkers in such sample. For example, in certain diseases it is important to understand how many cells, i.e. which fraction of the total number of cells, carry a certain biomarker. Such information is the basis for the staging and monitoring of numerous diseases, such as cancers, and has direct implications on the treatment to be employed.

Other applications for the detection of co-occurring mRNA species cells will be self apparent to the skilled artisan.

The present invention also provides for the detection of two or more DNA species in a cell. For example, the first DNA species may be a first gene or a gene fragment and the second DNA species may be a second gene or gene fragment. Such gene fragment may be single nucleotide polymorphisms (SNPs) or other genomic markers. Therefore, in accordance with the present invention the occurrence of two or more SNPs or other genomic markers can be detected. If a multitude of markers is detected, the present invention provides a method to simultaneously screen, detect or characterize DNA samples in any given sample, such as a sample from a patient. The methods of the present invention therefore provide a convenient way to characterize genetic material. Such information is useful in the diagnostic and the medical field. For example, the detection of certain resistance markers is a valuable parameter in deciding about different treatment options. In many leukemia and lymphoma the percentage of such resistance markers increases over time. The method of the present invention therefore provides a valuable tool to quantify said resistance markers, thereby indication an adequate treatment option. Similar other uses are possible, including the characterization and quantification of certain gene arrangements or rearrangements, such as the characterization of T cell receptors, complement, other variable parts of the immune system or gene mosaics.

In other aspects the methods of the present invention may be used to detect and characterize DNA methylation pattern. Such methylation patterns, likewise, are valuable markers for disease progression and treatment options.

The present invention also provides for the detection of two or more peptides, polypeptides or proteins in a cell. Like described for nucleic acids herein above, also peptides, polypeptides and proteins are indicative for certain diseases, disorders, conditions, or certain disease stages. Therefore, the simultaneous detection of two or more peptides, polypeptides or proteins is also a valuable tool for many applications.

In certain embodiments the present invention provides a method for the detection of the interaction of two or more biomolecules. In certain aspects the interaction of said biomolecules occurs in a cell. In other aspects the interaction of said biomolecules occurs outside a cell, on the cell surface or in a cell-free environment. FIG. 2 shows certain scenarios. For example, the method of the present invention may be employed to detect and identify cell-cell interactions, antibody-antigen interactions, e.g., the interaction of phage display libraries with an antigen, or the interaction of cells with other biomolecules, such as hormones, growth factors or other molecules.

### EXAMPLES

### Example 1: Coupling of the Storage and the Binder

Sequencing beads contain small adapter-ligated single strand DNA fragments of a specific sequence (hereinafter, sequence A). Exemplary beads are those which can be purchased for sequencing with the system from 454 Life Sciences (now a subsidiary of Roche). Alternatively, any other bead may be purchased and loaded with a DNA fragment of a specific sequence. Such a bead loaded with a small adapter-ligated single strand DNA fragment serves as storage.

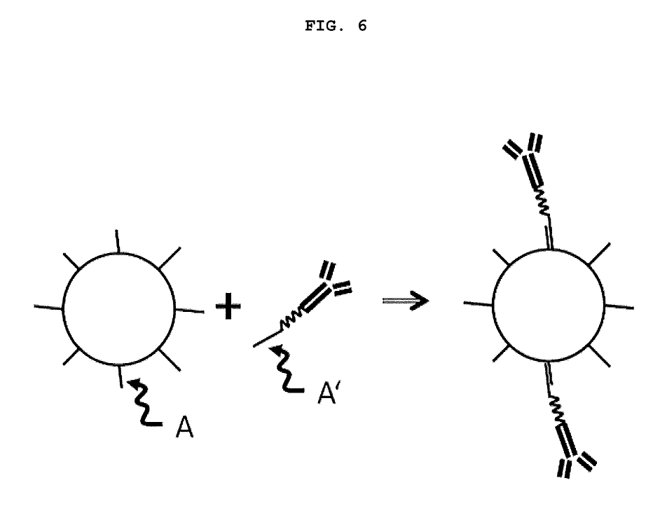

As a binder, an antibody is used which comprises at its C-terminus or its N-terminus a DNA fragment (sequence A) which is complementary to the DNA fragment of the sequencing bead (sequence A′). Such antibodies are commercially available or can be generated de novo. In this Example we use an antibody which is specific for B cells. This yields in beads which carry and present an antibody of choice (here: an antibody specific for B cells). The generation of such antibody-loaded beads is depicted in FIG. 6.

### Example 2: Capture of the Biological Entity

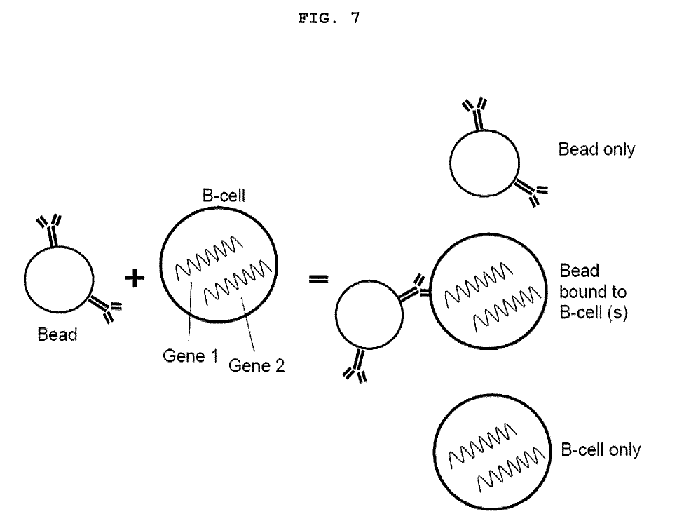

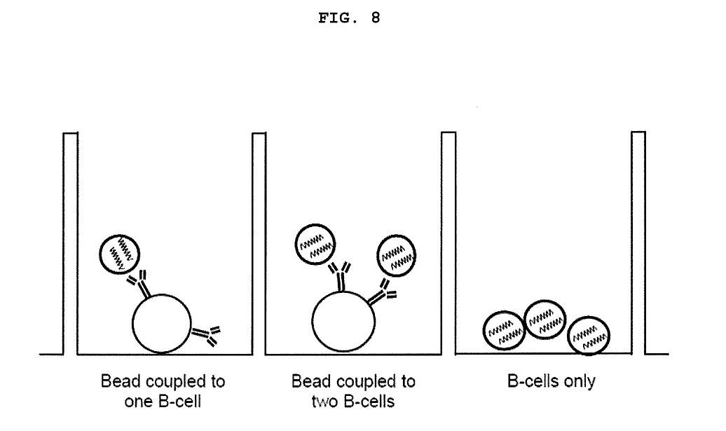

In this Example, B cells of an individual infected with a certain pathogen or having a certain disease or disorder are captured to the resulting beads from Example 1. The beads generated in Example 1 are specific for B cells and therefore bind to the B cells of a sample when incubated at the appropriate conditions. Since this is a stochastic process various products may form: empty beads, beads binding a single B cell, beads binding two or more B cells, isolated B cells (B cells which were not captured by any bead). This step is depicted in FIG. 7.

### Example 3: Generation of an Effective Range or a Compartment

The beads comprising the captured biological entity are filled into the cavities of a picotiterplate by centrifugation. Due to the size of the cavities, each cavity may contain no more than one single bead. Since this is also a stochastic process the following scenarios can occur: (1) a cavity contains a bead with a single cell, (2) a cavity contains a bead with two or more cells, (3) a cavity contains one or more cells but no beads and (4) a cavity is empty. This step is depicted in FIG. 8.

After the cavities were filled with the beads, the picotiterplate is washed and a PCR mix is added (for details see Example 4). A lid is added to generate an effective range or compartment. Either the bead, the walls of the picotiterplate or the lid may serve as storage.

Next, the biounits or biomolecules are released from the biological entity. This is achieved by lysing the cells at 95° C. The cells will burst and release the biounits or biomolecules. Also, the antibody will detach from the bead is now available for a sequencing reaction. The biounits or biomolecules of the present example are two genes, more specifically one gene encoding the variable heavy chain of an antibody (gene 1) and the gene encoding the variable light chain of the same antibody (gene 2).

### Example 4: Binding of the Biounits or Biomolecules to the Storage and Amplification

As outlined in Example 1 the bead contains a sequence A which is complementary to the sequence A′ on the antibody. The very same complementarity between A and A′ is used in the binding of the biounit or biomolecule of the present example to the storage.

The PCR mix added in Example 3 comprises a forward primer for gene 1 (P1), a reverse primer for gene 1 (R1), a forward primer for gene 2 (P2), and a reverse primer for gene 2 (R2).

Primer P1 contains three regions:
Sequence A′, which is complementary to sequence on the bead
Sequence C′, which is subsequently used for sequencing
Sequence X′, which is complementary to sequence X of gene 1.

Primer R1 is complementary to the region R1′ of gene 1.

PCR amplification in the cavity of the picotiterplate leads to a product A-C-X-gene 1-R1.

Primer P2 contains three regions:
Sequence A′, which is complementary to sequence on the bead
Sequence D′, which is subsequently used for sequencing
Sequence Y′, which is complementary to sequence Y of gene 2.

Primer R2 is complementary to the region R2′ of gene 2.

PCR amplification in the cavity of the picotiterplate leads to a product A-D-Y-gene 2-R2.

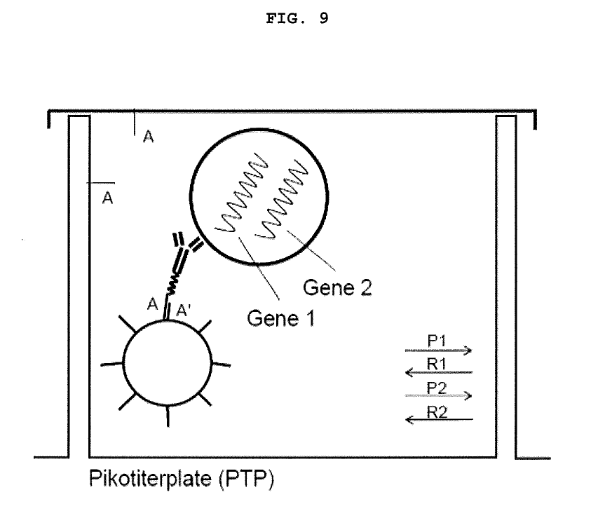

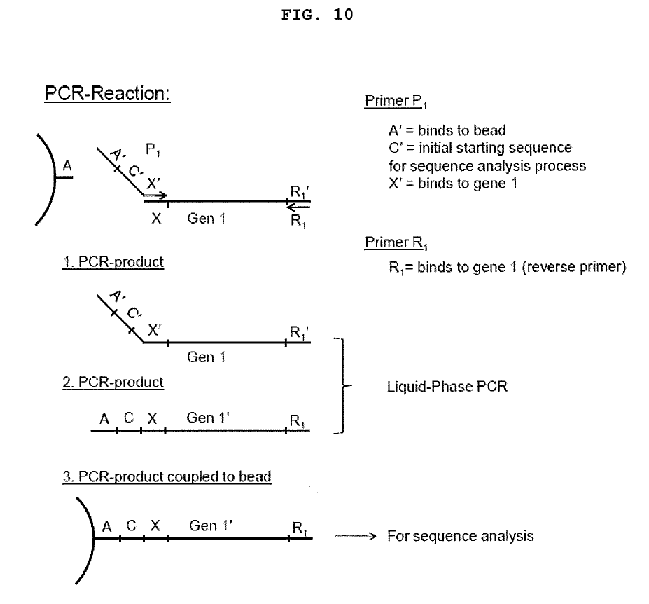

This step is depicted in FIG. 9, FIG. 10 and FIG. 11.

### Example 5: Detecting the Biounits or Biomolecules

A standard sequencing reaction is performed using sequencing primer C′, which is complementary to the region C of gene 1. The nucleic acid sequence of the entire nucleic acid strand is now determined with standard technology, e.g., pyrosequencing with a 454 sequencer. Likewise gene 2 is sequenced utilizing primer D′, which is complementary to the region D of gene 2.

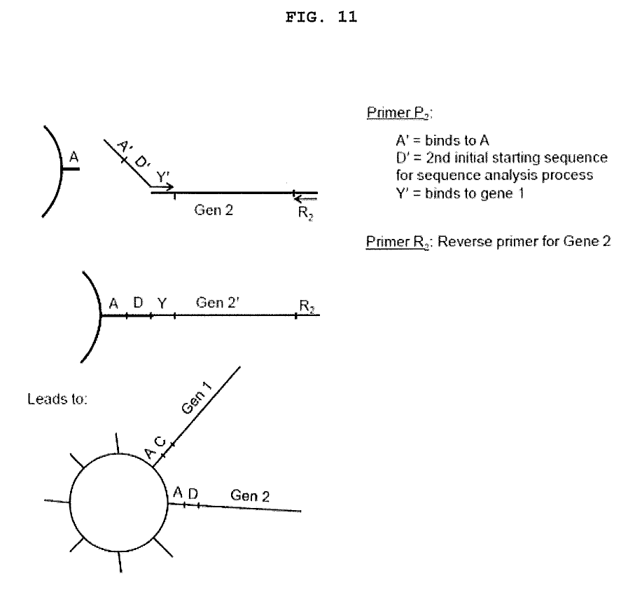

This step is depicted in FIG. 12.

### Example 6: Elimination of Sequence Data from Cavities Comprising More than One Biological Entity or Cell

As described in Example 3, a cavity may contain a bead with two or more cells. Such cavities will produce sequencing data (Example 4) which cannot be readily interpreted, since sequencing will deliver mixed signals, i.e. signals derived from two, or even more, nucleic acid molecules of different sequences. Such cavities may be identified by incorporation a calibration sequence into primer used for sequencing. The calibration sequence is located between the sequence A′, i.e. the part of the primer which is complementary to sequence on the bead, and the sequence X′ (or Y′), which is complementary to sequence X (Y) of gene 1 (gene 2). In its easiest form the calibration sequence only contains the four different nucleotides A, C, G and T. It may however also comprise additional, redundant nucleotides of any order, whereas it is preferred that each of the four nucleotides occurs at least once within the calibration sequence.

During sequencing, each nucleic acid molecule starts with the same calibration sequence, even if the individual molecules carry different subsequent genes, i.e. if there are different genes X in the PCR mix. This situation will occur, if the cavity contains a bead with two or more cells.

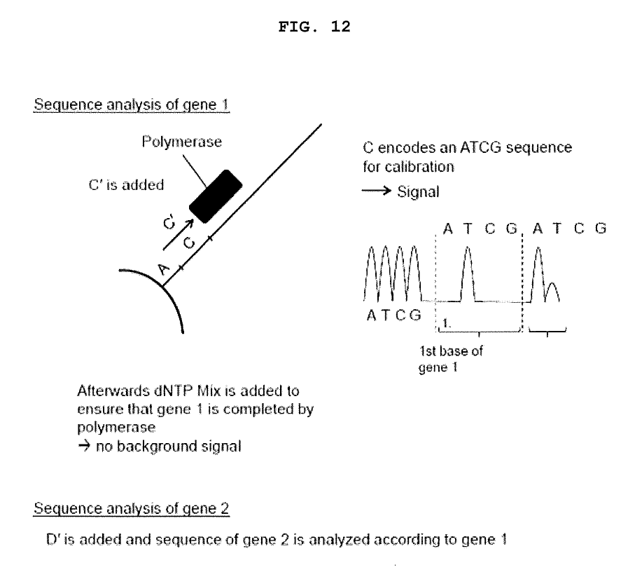

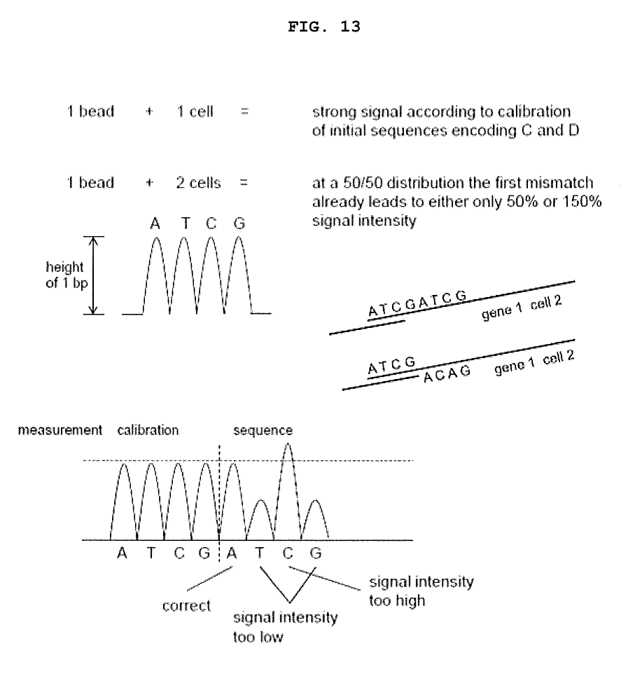

Such cavities can be identified since later in the sequencing process the signals will differ from those obtained with the calibration sequence, i.e. the sequencing signals will be lower than those obtained with the calibration sequence and there will be mixed signals, i.e. signals for more than one nucleotide will be obtained. Such cavities can be identified and exempted from further analysis. The process is depicted in FIG. 13.

### Example 7: Emulsion in Oil

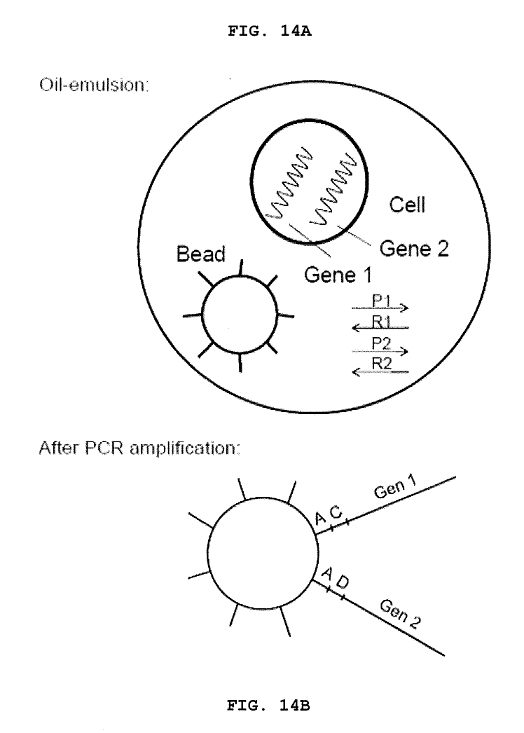

Rather than using a picotiterplate, the method of the present invention can also be practiced using an emulsion of water in oil. To do so Examples 1 and 2 are performed as described herein above. Instead of continuing with Example 3, an oil emulsion is prepared. The water droplets comprise the beads with the cells and the PCR mixture. The phase boundary between the water and the oil generates and defines the effective range. See FIG. 14A and FIG. 14B. Binding of the biounits or biomolecules to the storage, amplification and detection is done as described in Examples 4 and 5. Instead of a standard PCR an emulsion PCR is performed. This embodiment can also be practiced with a calibration sequence (see Example 6).

### Example 8: Library-Versus-Library Screening

In this example we describe the technological approach to screen a first library against a second library. It is thereby possible to identify all interactions between the biounits or biomolecules contained in the first library with the biounits or biomolecules contained in the second library.

A first phage library comprises a gene library, wherein each nucleic acid encoding a gene of the library contains at least the following three regions:
Sequence C, which is subsequently used for sequencing
Sequence X, which encodes for gene X of the library, and
Sequence R1, which is also subsequently used for sequencing.

The phages of the first library also contain a tag on its surface. This tag can be any entity, preferably a peptide sequence that can be used to capture and isolated the phages carrying such tag. Such a tag may be any epitope which is recognized by a respective antibody. This antibody comprises at its C-terminus a DNA fragment which is complementary to a DNA fragment on a sequencing bead (sequence A′). See Example 1 for more details.

A second phage library comprises a gene library, wherein each nucleic acid encoding a gene of the library contains at least the following three regions:
Sequence D, which is subsequently used for sequencing
Sequence Y, which encodes for gene Y of the library, and
Sequence R2, which is also subsequently used for sequencing.

In a first step the two phage libraries expressing the gene products of interest are mixed under conditions that allow to form interactions between the gene products of the first library with the gene products of the second library. After the respective phage pairs are formed, an antibody with specificity for the tag presented on the first phage library is added. Via its C-terminal sequence this antibody will bind to the corresponding sequence on a bead, thereby forming a complex comprising a bead, an antibody and two phages. Since this is however a stochastic process various products may form (not listing the antibody, since it is only used as a technological vehicle): (1) empty beads, (2) beads containing a phage of the first library, (3) beads containing a phage of the first and a phage of the second library, and (4) beads containing more than one phages of the first library and/or more than one phages of the second library.

PCR sequencing is performed utilizing primers P1 and R1 for gene X and primers P2 and R2 for gene Y. Primer P1 contains two regions: sequence A′, which is complementary to sequence on the bead, and sequence C. Primer R1 contains sequence R1′ which is complementary to sequence R1. Primer P2 contains two regions: sequence A′, which is complementary to sequence on the bead, and sequence D. Primer R2 contains sequence R2′ which is complementary to sequence R2.

The following sequences will be obtained, depending on the products formed recited above:

case (1) empty beads: no signal, i.e. no sequence

case (2) bead+phage 1: correct sequence, but only for phage of the first library, i.e. no interaction partner

case (3) bead+phages 1&2: correct sequence of the two interacting polypeptides

case (4) bead with several phages: mixed signals, no interpretation possible

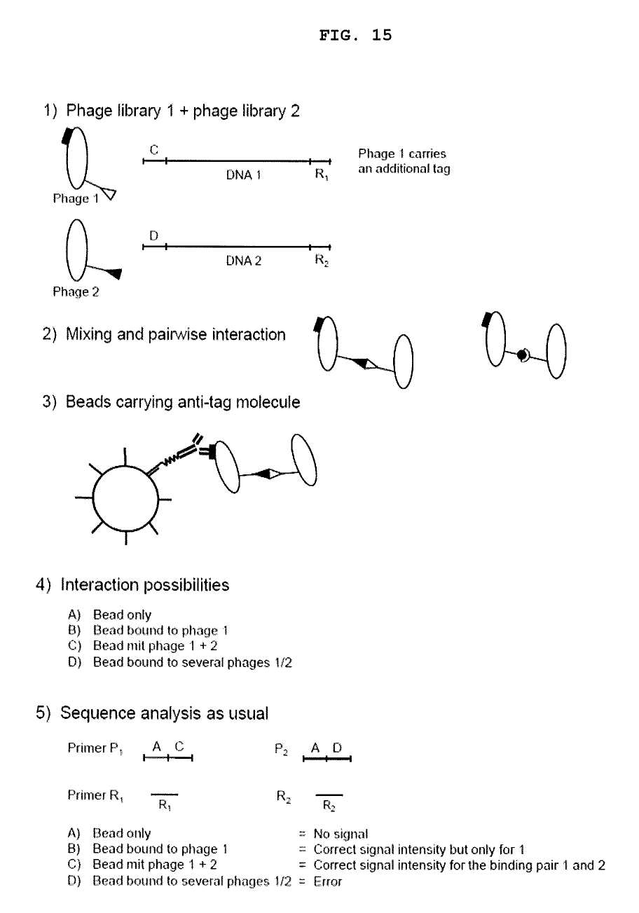

FIG. 15 outlines the library-versus-library screening approach.

### Example 9: An Improved Yeast-Two-Hybrid (Y2H) System

The yeast two hybrid system (Fields & Song, Nature (1989), 340, 245-6) is used for the identification of interacting proteins. The key principle is that a part of a transcription factor in the original publication GAL4-BD is fused to a protein, the so called bait, in a reporter yeast strain (containing lacZ upstream of the UAS promoter (activated by the intact GAL protein), this reporter yeast is transformed with a library of potential interaction partners fused two a second domain of the GAL protein named GAL4-AD), the so called prey library. If the prey and the bait protein interact a functional GAL protein is assembled and transcription of lac Z takes place, leading to release of a blue precipitate of X-gal (5-bromo-4-chloro-3-indolyl-β-D-galactoside), which is added to the growth agar. The blue colonies are picked and the prey sequence is analyzed and identified as potential interaction partner for the bait sequence. In an improved method Joung et al (PNAS (2000) 97, 7382-7) developed a reporter system based on spectinomycin resistance conferred by the product of the HIS3 gene. The method described within the present invention will improve this method massively. The method described would allow yeast two hybrid library versus. library in high throughput and a yet unseen signal-to-background and signal-to-noise ratio. A bait library is transformed into a yeast strain and this library is co-transformed with the prey library. Then a positive selection system, e.g., the system described by Joung et al. is applied (e.g., in liquid culture). Yeast cells are isolated and sequenced while maintaining respective prey/bait pairings.

The bait library comprises a gene library in yeast where e.g., the HIS3 gene is under control of the UAS promoter, wherein each nucleic acid encoding a gene of the library contains at least the following three regions:
Sequence C, which is subsequently used for sequencing
Sequence X, which encodes for gene X of the library fused to the respective part of the bait regulatory sequence, e.g., (GAL4-BD)
Sequence R1, which is also subsequently used for sequencing.

A second library (plasmid encoded) encodes the prey gene library, wherein each nucleic acid encoding a gene of the library contains at least the following three regions:
Sequence D, which is subsequently used for sequencing
Sequence Y, which encodes for gene Y of the library fused to the complementary part of the transcription factor (e.g., GAL4-AD), and
Sequence R2, which is also subsequently used for sequencing.

The yeast bait library is co-transformed with the prey library and grown under the respective positive selection conditions (e.g., in the presence of spectinomycin). Growing yeast cells are immobilized on beads using limited dilution techniques. PCR sequencing is performed utilizing primers P1 and R1 for gene X and primers P2 and R2 for gene Y. Primer P1 contains two regions: sequence A′, which is complementary to sequence on the bead, and sequence C. Primer R1 contains sequence R1′ which is complementary to sequence R1. Primer P2 contains two regions: sequence A′, which is complementary to sequence on the bead, and sequence D. Primer R2 contains sequence R2′ which is complementary to sequence R2.

The following sequences will be obtained, depending on the products formed recited above:

case (1) empty beads: no signal, i.e. no sequence

case (2) bead+bait yeast: correct sequence, but only for bait, false positive should not happen due to positive selection pressure.

case (3) bead+baid and prey: correct sequence of the two interacting polypeptides

case (4) bead with several prey sequences: probably caused by immobilizing more than one yeast.

What is claimed:

1. A method for the detection of two or more biomolecules, said method comprising
(a) providing a sample comprising a cell comprising said biomolecules,
(b) spatially separating said cell in a compartment comprising a moiety which is able to bind derivatives of said biomolecules,
(c) releasing the biomolecules from the cell,
(d) generating derivatives of said biomolecules,
(e) allowing the derivatives of said biomolecules to bind to the moiety which is able to bind the derivatives of said biomolecules, and
(f) detecting or identifying the derivatives of the biomolecules.

2. The method of claim 1, wherein said biomolecules are selected from the group consisting of the sub-classes polypeptides, proteins, peptides, nucleic acids, carbohydrates, fatty acids, small molecules, cell organelles, or derivatives, parts or combinations of any of the foregoing.

3. The method of claim 2 wherein all biomolecules are from the same subclass.

4. The method of claim 3, wherein said subclass is the subclass of polypeptides or the subclass of nucleic acids.

5. The method of claim 4, wherein each of said polypeptides is part of a multimeric protein or enzyme or wherein each of said nucleic acids encodes for a polypeptide which is part of a multimeric protein or enzyme.

6. The method of claim 5, wherein said multimeric protein is an immunoglobulin, or a functional fragment thereof.

7. The method of claim 1, wherein said biomolecules are genes encoding the variable heavy and the variable light chain of an immunoglobulin or a functional fragment thereof.

8. The method of claim 2 wherein the biomolecules are from different subclasses.

9. The method of claim 1, wherein said sample is or is derived from blood, bone marrow, a tumor, a single cellular organism, a prokaryote or a body fluid.

10. The method of claim 9, wherein said sample is a sample from a patient, wherein said patient is a healthy patient, an immunized patient, an infected patient or a patient with a disease or disorder.

11. The method of claim 1 wherein said cell is a single cell, such as a single B cell.

12. The method of claim 1, wherein said cell is a cell interacting with another cell, a virus, a bacterium, a molecule or a biomolecule, or derivatives, fragments or composites of any of the foregoing.

13. The method of claim 1, wherein said cell comprises a mixture of two chemical and/or biological libraries, wherein at least one member of the first library interacts with or binds to a member of the second library.

14. The method of claim 1, wherein said cell comprises a biomolecule which interacts with or binds to at least two members of a chemical and/or biological library.

15. The method of claim 1 wherein said compartment is formed by a cavity, a well, an emulsion, a phase-boundary-system, a hydrophobic spot, a particle, physical forces or chemical cross-linking.

16. The method of claim 15, wherein said phase boundaries are realized by a phase separation between water and gas like water droplets in air or water and a liquid like water droplets in oil or water and a solid phase like water droplets in a microtiterplate.

17. The method of claim 15, wherein said cavity or said well is a cavity on a microtiterplate, a picotiterplate or a microstructured substrate.

18. The method of claim 15, wherein said emulsion is a water-in-oil or an oil-in-water emulsion.

19. The method of claim 15, wherein said particle consists of silica, glass, agarose, a polymer, a metal oxide or a composite thereof.

20. The method of claim 15, wherein said physical forces are electrostatic forces, electrodynamic forces, dielectrophoretic forces, electromagnetic forces, magnetic, optical, temperature or density effects.

21. The method of claim 1, wherein said moiety which is able to bind derivatives of said biomolecules is a bead, a glass slide, a microtiterplate, a picotiterplate, or a lid of any of the foregoing.

22. The method of claim 1 wherein step (c) is performed by a change of the chemical or physical conditions.

23. The method of claim 22, wherein the change of chemical conditions is a pH change, a change of salt concentrations, the addition of an enzyme, the addition of lytic agents.

24. The method of claim 22, wherein the change of physical conditions is heating, freezing, application of electric, magnetic or dielectric fields, sheer or centrifugal forces, mechanical deformation, relaxation, ultrasonic or any physical disruptive effect.

25. The method of claim 24, wherein said change of the physical condition is effected in a time dependent manner, such as dissolving of a particle in a solution, the dissolving of a protective shell around the biounit or the induction by an enzyme.

26. The method of claim 1 wherein step (d) includes an amplification reaction which leads to the generation of replicates or derivatives of said biounits.

27. The method of claim 26, wherein said amplification reaction is a PCR or a RT-PCR, and wherein during said PCR or RT-PCR a [first] tag is added which enables said replicates or derivatives to bind to the moiety which is able to the derivatives of said biomolecules.

28. The method of claim 27, wherein during said PCR or RT-PCR a second tag is added which enables subsequent sequencing of the PCR or RT-PCR product.

29. The method of claim 1 wherein step (e) is performed by DNA sequencing.

30. The method of claim 29, wherein said DNA sequencing is performed by sequencing the PCR or RT-PCR products sequentially or in parallel.

31. The method of claim 29, wherein said DNA sequencing is performed by sequencing the PCR or RT-PCR products on the moiety which is able to bind the PCR or RT-PCR products or on copies of said moiety.

32. The method of claim 1, wherein said biomolecules are nucleic acids that bind by hybridization to a moiety which is able to bind said nucleic acid, wherein said moiety is a solid-phase particle, and wherein said solid-phase particle is used for sequencing in step (e).

33. The method of claim 1, wherein said biomolecules are polypeptides or proteins that bind directly to the surface of the moiety which is able to bind said polypeptides or proteins, wherein said moiety is a solid-phase particle, and wherein said biomolecules on said solid-phase particle is detected via an immunoassay in step (e).

34. The method of claim 1, wherein the detecting or identification of the biomolecules or their derivatives is performed simultaneously.

35. The method of claim 1, wherein said sample comprises at least 10³, at least 10⁶, at least 10⁹ or at least 10¹² cells, and wherein in each of said cells at least two biomolecules are detected.

36. The method of claim 35, wherein the correlation of the presence of said at least two subunits within said cells is statistically analyzed or determined.

37. An immunoassay incorporating or utilizing the method of claim 1.

38. A device for performing a method of claim 1.

39. A kit comprising a device and instruction to perform the method of claim 1.
